(19) United States
(12) Patent Application Publication
(10) Pub. No.: US 2005/0228180 A1
(43) Pub. Date: Oct. 13, 2005

(54) Alpha, omega-dicarboximide derivatives as useful uro-selective a1a adrenoceptor blockers

(76) Inventors: Mohammad Salman, Haryana (IN); Gyan Chand Yadav, Ghaziabad (IN); Somesh Sharma, New Delhi (IN); Gobind Singh Kapkoti, Haryana (IN); Anita Chugh, New Delhi (IN); Jang Bahadur Gupta, Haryana (IN); Nitya Anand, Lucknow (IN)

Correspondence Address: RANBAXY INC., 600 COLLEGE ROAD EAST, SUITE 2100, PRINCETON, NJ, 08540, US

(21) Appl. No.: 10/510,362

(22) PCT Filed: Apr. 8, 2002
(86) PCT No.: PCT/IB02/01113

§ 371 Date: May 25, 2005

(51) Int. Cl. A61K 31/496; C07D 43/02

(52) U.S. Cl. 544/360; 544/373; 514/253.1; 514/254.08

(57) ABSTRACT

Novel α,β-dicarboximide derivatives which selectively inhibit binding to the α- [symbol] ,1A? adrenergic receptor, a receptor which has been shown to be important in the treatment of benign prostatic hyperplasia. The compounds of the present invention are potentially useful in the treatment of benign prostatic hyperplasia.

### FIELD OF THE INVENTION

This invention relates to certain novel α,β-dicarboximide derivatives which selectively inhibit binding to the α_{1A} adrenergic receptor, a receptor which has been shown to be important in the treatment of benign prostatic hyperplasia. The compounds of the present invention are potentially useful in the treatment of benign prostatic hyperplasia. This invention also relates to methods for synthesizing the novel compounds, pharmaceutical compositions containing the compounds, methods of treating benign prostatic hyperplasia using the compounds, and intermediate compounds used in the preparation of novel compounds.

### BACKGROUND OF THE INVENTION

Benign prostatic hyperplasia (BPH), a nonmalignant enlargement of the prostate, is the most common benign tumor in men. Approximately 50% of all men older than 65 years have some degree of BPH and a third of these men have clinical symptoms consistent with bladder outlet obstruction (Hieble and Caine, Fed. Proc., 1986; 45:2601). Worldwide benign and malignant diseases of the prostate are responsible for more surgery than diseases of any other organ in men over the age of fifty.

It is generally accepted that there are two components of BPH, a static and a dynamic component. The static component is due to enlargement of the prostate gland, which may result in compression of the urethra and obstruction to the flow of urine from the bladder. The dynamic component is due to increased smooth muscle tone of the bladder neck and the prostate itself (which interferes with emptying of the bladder) and is regulated by alpha 1 adrenergic receptors (α₁-ARs). The medical treatments available for BPH address these components to varying degrees, and the therapeutic choices are expanding.

Surgical treatment options address the static component of BPH and include transurethral resection of the prostate CFURP), open prostatectomy, balloon dilatation, hyperthermia, stents and laser ablation. Although, TURP is the gold standard treatment for patients with BPH, approximately 20-25% of patients do not have a satisfactory long-term outcome (Lepor and Rigaud, J. Urol., 1990; 143:533). Postoperative urinary tract infection (5-10%), some degree of urinary incontinence (2-4%), as also reoperation (15-20%) (Wennberg et al., JAMA, 1987; 257:933) are some of the other risk factors involved.

Apart from surgical approaches, there are some drug therapies which address the static component of this condition. Finasteride (Proscar, Merck), is one such therapy which is indicated for the treatment of symptomatic BPH. This drug is a competitive inhibitor of the enzyme 5α-reductase which is responsible for the conversion of testosterone to dihydrotestosterone in the prostate gland (Gormley et al., N. Engl. J. Med., 1992; 327:1185). Dihydrotestosterone appears to be the major mitogen for prostate growth, and agents which inhibit 5-α-reductase reduce the size of the prostate and improve urine flow through the prostatic urethra. Although finasteride is a potent 5α-reductase inhibitor and causes a marked decrease in serum and tissue concentration of dihydrotestosterone, it is only moderately effective in treating symptomatic BPH (Oesterling, N. Engl. J. Med., 1995; 332:99). The effects of finasteride take 6-12 months to become evident and for many men the clinical improvement is minimal.

Due to the limited effectiveness of 5α-reductase inhibitors in terms of immediate symptomatic and urodynamic relief, other pharmacological approaches have been assessed in the clinical setting.

The dynamic component of BPH has been addressed by the use of adrenergic receptor blocking agents (α₁-AR blockers) which act by decreasing the smooth muscle tone within the prostate gland itself. α₁-adrenergic receptor antagonists appear to be much more effective and provide immediate subjective symptomatic improvements and are, therefore, the preferred modalities of treatment in the control of benign prostate hypertrophy. α₁-Adrenoceptors are also present in blood vessels and play an important role in the regulation of blood pressure. Thus, α₁-adrenoceptor antagonists are of particular importance as they were originally developed as antihypertensive agents and are likely also to have a beneficial affect on lipid dysfunction and insulin resistance, which are commonly associated with essential hypertension.

The use of α₁-AR antagonists in the treatment of BPH is related to their ability to decrease the tone of prostatic smooth muscle, leading to relief of the obstructive symptoms. Adrenergic receptors found throughout the body play a dominant role in the control of blood pressure, nasal congestion, prostate function and other processes (Harrison et al., Trends Pharmacol. Sci., 1991; 12:62). There are a number of cloned α₁-AR receptor subtypes: α_{1A}-AR, α_{1B}-AR and α_{1D}-AR (Bruno et al., Biochem. Biophys. Res. Commun., 1991; 179:1485; Forray et al., Mol. Pharmacol., 1994; 45:703; Hirasawa et al., Biochem. Biophys. Res. Commun., 1993; 195:902; Ramarao et al., J. Biol. Chem., 1992; 267:21936; Schwinn et al., JPET, 1995; 272:134; Weinberg et al., Biochem. Biophys. Res. Commun., 1994; 201:1296). A number of laboratories have characterized the α₁-ARS in human prostate by function, radioligand binding, and molecular biological techniques (Forray et al., Mol. Pharmacol. 1994; 45:703; Hatano et al., Br. J. Pharmacol, 1994; 113:723; Marshall et al., Br. J. Pharmacol. 1992; 112:59; Marshall et al., Br. J. Pharmacol., 1995; 115:781; Yamada et al., Life Sci., 1994; 54:1845). These studies provide evidence in support of the concept that the α_{1A}-AR subtype comprises the majority of α₁-ARS in human prostatic smooth muscle and mediates contraction in this tissue. These findings suggest that the development of a subtype-selective α_{1A}-AR antagonists might result in a therapeutically effective agent with reduced side effects for the treatment of BPH.

A variety of α₁-AR blockers (terazosin, prazosin, and doxazosin) have been investigated for the treatment of symptomatic bladder outlet obstruction due to BPH, with terazosin (Hytrin, Abbott) being the most extensively studied. Although the α₁-AR blockers are well tolerated, approximately 10-15% of patients develop a clinically adverse event The undesirable effects of all members of this class are similar, with postural hypotension being the most commonly experienced side effect.

The α₁-AR blocking agents have a more rapid onset of action. However, their therapeutic effect, as measured by improvement in the symptom score and the peak urinary flow rate, is moderate. (Oesterling, N. Engl. J. Med., 1995; 332:99). The vascular side effects (e.g., postural hypertension, dizziness, headaches, etc.) associated with these drugs is due to lack of selectivity of action between prostatic and vascular α₁-adrenoceptors. Clearly, α₁-adrenoceptor antagonists which have inherently greater selectivity for prostatic α₁-adrenoceptors offer the potential of increased urodynamic benefits. This underscores the importance of the discovery of prostate-selective α₁-adrenoceptor antagonists which will confer urodynamic improvement without the side effects associated with existing drugs.

There are many description in the literature about the pharmacological activities associated with α, ω-dicarboximide derivatives. Eur. J. Med. Chem. Chemica Therapeutica; 1977; 12(2):173, J. Indian. Chem. Soc., 1978; LV:819; J. Indian Chem. Soc., 1979; LVI:1002 discuss the synthesis of these derivatives with CNS and antihypertensive activity. Other references like U.S. Pat. Nos. 4,524,206; 4,598,078; 4,567,180; 4,479,954; 5,183,819; 4,748,240; 4,892,943; 4,797,488; 4,804,751; 4,824,999; 4,957,913; 5,420,278; 5,330,762; 4,543,355 and PCT application Nos. WO 98/37893; WO 93/21179, also describe CNS and antihypertensive activity of these compounds. There is no mention of adrenoceptor blocking activity of these compounds and thus their usefulness in the treatment of BPH did not arise.

J. Med. Chem., 1983; 26:203 reports dopamine and α₁-adrenergic activity of some Buspirone analogues. EP 078800 discusses α₁-adrenergic receptor antagonistic activity of pyrimidinedione, pyrimidinetrione and triazinedione derivatives. These compounds, however, had low α₁-adrenergic blocking activity as compared to known α₁-antagonists.

The earlier synthesis of various 1-(4-arylpiperazin-1-yl)-3-(2-oxo-pyrrolidin-1-yl/piperidin-1-yl)alkanes and their usefulness as hypotensive and antischemic agents is disclosed in Indian Patent applications 496/DEL/95, 500/DEL/95 and 96/DEL/96. These compounds had low α₁-adrenergic blocking activity (pKi˜6 as compared to >8 of the known α₁-antagonists such as prazosin), and practically no adrenoceptor sub-class selectivity for CLIA VS. α_{1B} or α_{1D} adrenoceptors. Further work showed that structural modification of these compounds from lactam to dioxo compounds, i.e., from 2-oxopyrrolidin to 2,5-dioxopyrrolidin and 2,6-dioxopiperidine, enhances the adrenoceptor blocking activity, and also greatly increases the selectivity for α_{1A} in comparison to α_{1B}-adrenoceptor blocking activity, an essential requirement for compounds to be good candidates for treatment of benign prostatic hyperplasia (BPH) disclosed in our U.S. Pat. Nos. 6,083,950 and 6,090,809 which are incorporated herein by reference.

### OBJECTS OF THE INVENTION

Recently, it has been demonstrated that the prostate tissue of higher species like man and dog has a predominant concentration of α_{1A}-adrenoceptor subtype. This makes it possible to develop agents with selective action against these pathological urodynamic states. The present invention is directed to the development of novel α₁-adrenoceptors and which would thus offer a viable selective relief for prostate hypertrophy as well as essential hypertension, without the side effects associated with known α_{1A}-AR antagonists.

The objective of the present invention therefore is to provide novel α,ω-dicarboximide derivatives that exhibit significantly greater α_{1A}-adrenergic blocking potency than available with known compounds in order to provide specific treatment for benign prostatic hyperplasia.

It is also an object of the invention to provide a process for synthesis of the novel compounds.

It is a further object of the invention to provide compositions containing the novel compounds which are useful in the treatment of benign prostatic hyperplasia.

### SUMMARY OF TE INVENTION

In order to achieve the above mentioned objectives and in accordance with the purpose of the invention as embodied and described herein, there are provided novel α, ω-dicarboximide derivatives represented by Formula I below; 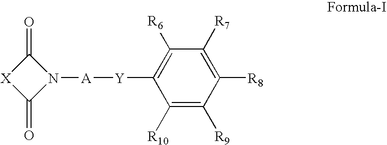
wherein X is selected from the group consisting of 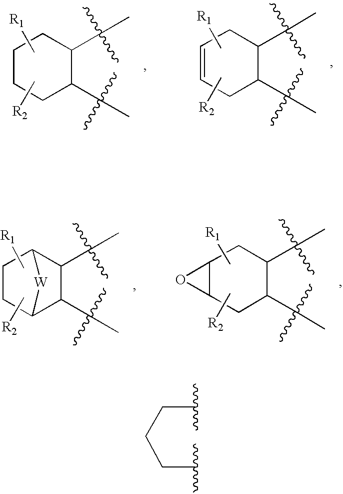
where the points of attachment are depicted by hashed bonds, and where one point of attachment is bonded to the carbonyl adjacent to the nitrogen and the second point of attachment is bonded to the other carbonyl;
W is O, S, SO or SO₂; 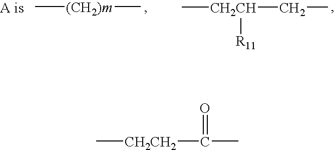
   where m is one of the integers 2, 3 or 4;
R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 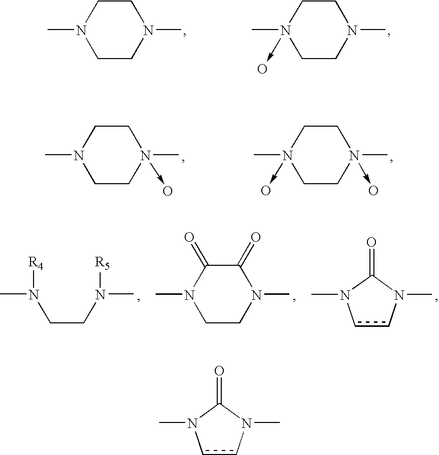
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄ alkylthio, lower (C₁₋₄) perhaloalkyl, lower (C₁₋₄) perhaloalkoxy lower (C₁₋₄) alkoxy substituted with one or more of F, Cl, Br, I, OH, or OR₃, or optionally substituted groups selected from aryl, aralalkyl, heterocyclyl or heteroaryl, said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl, optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower (C₁-C₄) alkyl amino carbonyl, hydroxy substituted with aromatic or non-aromatic five or six membered ring, phenyl or phenyl substituted by Cl, F, Br, I, NO₂, NH₂, (C₁₋₄) alkyl or (C₁₋₄) alkoxy, (C₁₋₄) perhaloalkyl, (C₁₋₄) perhaloalkoxy wherein the broken line 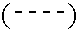
   is a single bond or no bond.

The present invention also provides pharmaceutical compositions for the treatment of benign prostatic hyperplasia. These compositions comprise an effective amount of at least one of the above compounds of Formula I and/or an effective amount of at least one physiologically acceptable acid addition salt thereof, with a pharmaceutically acceptable carrier.

An illustrative list of particular compounds of the invention is given below:

1-[4-(2-Hydroxyphenyl)piperazin-1-yl]-3-(2,6-dioxopiperidin-1-yl)propane hydrochloride; (Compound No. 1)
2-[3-{4-(2-(2,2,2-Trifluoroethoxy)phenyl)piperazin-1-yl}propyl]-3a, 4,7,7a-tetrahydro-1H-isoindole-1,3(2H)-dione hydrochloride; (Compound No. 2)
1-[4-{2-(2,2,2-Trifluoroethoxy)phenyl piperazin-1-yl]-3-(2,6-dioxopiperidin-1-yl)propanehydrochloride; (Compound No. 3)
2-[3-{4-(2-Ethoxyphenyl)piperazin-1-yl, 1-N-oxide}propyl]-3a,4,7,7a-tetrahydro-1H-isoindole-1,3(2H)-dione; (Compound No. 4)
1-[4-(2-Ethoxyphenyl)piperazin-1-yl]-3-(2,6-dioxopiperadin-1-yl)ethane hydrochloride; (Compound No. 5)
2-[3-{4-(2-Methoxyphenyl)piperazin-1-yl}-2-hydroxypropyl]-3a,4,7,7a-tetrahydro-1H-isoindole-1,3(2H)-dione hydrochloride; (Compound No. 6)
2-[3-{4-(2-Isopropoxyphenyl)piperazin-1-yl}-2-hydroxypropyl]-3a,4,7,7a-tetrahydro-1H-isoindole-1,3(2H)-dione hydrochloride; (Compound No. 7)
2-[3-{4-(2-Isopropoxyphenyl)piperazin-1-yl, 1-N-oxide}propyl]-3a,4,7,7a-tetrahydro-1H-isoindole-1,3(2H)-dione; (Compound No. 8)
2-[3-{4-(2-Isopropoxyphenyl)piperazin-1-yl, 1,4-N,N-dioxide}propyl]-3a,4,7,7a-tetrahydro-1H-isoindole-1,3-(2H)-dione; (Compound No. 9)
2-[3-{4-(2-Ethoxyphenyl)piperazin-1-yl, 1,4-N,N-dioxide}propyl]-3a,4,7,7a-tetrahydro-1H-isoindole-1,3(2H)-dione; (Compound No. 10)
2-[3-{4-(2-Ethoxyphenyl)piperazin-1-yl}-2-hydroxypropyl]-3a,4,7,7a-tetrahydro-1H-isoindole-1,3-(2H)-dione hydrochloride; (Compound No. 11)
2-[3-{4-(2-Ethoxyphenyl)piperazin-1-yl}propyl]-5,6-dihydroxy-3a,4,5,6,7,7a-hexahydro-1H-isoindole-1,3(2H)-dione; (Compound No. 12)
2-[3-{4-(2-Isopropoxyphenyl)piperazin-1-yl}propyl]-5,6-dihydroxy-3a,4,5,6,7,7a-hexahydro-1H-isoindole-1,3(2H)-dione; (Compound No. 13)
2-[3-{4-(2-Hydroxyphenyl)piperazin-1-yl}-2-hydroxypropyl]-3a,4,7,7a-tetrahydro-1H-isoindole-1,3(2H)-dione hydrochloride; (Compound No. 14)
2-[2-{4-(2-Ethoxyphenyl)piperazin-1-yl}ethyl]-3a,4,7,7a-tetrahydro-1H-isoindole-1,3(2H)-dione hydrochloride; (Compound No. 15)
2-[2-{4-(2-(2,2,2-Trifluoroethoxy)phenyl)piperazin-1-yl}ethyl]-3a,4,7,7a-tetrahydro-1H-isoindole-1,3(2H)-dione hydrochloride; (Compound No. 16)
2-[3-{-(2-Ethoxyphenyl)piperazin-1-yl}propyl]-5-chloro-6-hydroxy-3a,4,5,6,7,7a-hexahydro-1H-isoindole-1,3(2H)-dione hydrochloride; (Compound No. 17)
2-[3-{4-(2-Ethoxyphenyl)piperazin-1-yl}propyl]-5-hydroxy-3a,4,5,6,7,7a-hexahydro-1H-isoindole-1,3(2H)-dione hydrochloride; (Compound No. 18)
2-[3-{4-(2-Isopropoxyphenyl)piperazin-1-yl}propyl]-5,6-epoxy-3a,4,5,6,7,7a-hexahydro-1H-isoindole-1,3(2H)-dione; (Compound No. 19)
2-[3-{4-(2-Isopropoxyphenyl)piperazin-1-yl}propyl]-5-hydroxy-3a,4,5,6,7,7a-hexahydro-1H-isoindole-1,3(2H)-dione; (Compound No. 20)
2-[3-{4-(2-Isopropoxy-5-hydroxyphenyl)piperazin-1-yl}propyl]-3a,4,7,7a-tetrahydro-1H-isoindole-1,3(2H)-dione hydrochloride; (Compound No. 21)
2-[3-{4-(2-Hydroxyphenyl)piperazin-1-yl, 1-N-oxide}propyl]-3a,4,7,7a-tetrahydro-1H-isoindole-1,3(2H)-dione; (Compound No. 22)
2-[3-{4-(2-Isopropoxyphenyl)piperazin-1-yl}propyl]-5,6-dihydroxy-3a,4,5,6,7,7a, hexahydro-1H-isoindole-1,3(2H)-dione-hydrochloride; (Compound No. 23)
2-[3-{4-(2-Ethoxy-5-hydroxyphenyl)piperazin-1-yl}propyl]-3a,4,7,7a-tetrahydro-1H-isoindole-1,3-(2H)-dione hydrochloride; (Compound No. 24)
2-[3-{4-(2-Isopropoxy-4-nitrophenyl)piperazin-1-yl lpropyl]-3a,4,7,7a-tetrahydro-1H-isoindole-1,3(2H)-dione hydrochloride; (Compound No. 25)
2-[3-{4-(2-Isopropoxy-4-aminophenyl)piperazin-1-yl}propyl]-3a,4,7,7a-tetrahydro-1H-isoindole-1,3(2H)-dione hydrochloride; (Compound No. 26)
2-[3-{4-(2-isopropoxy-6-hydroxyphenyl)piperazin-1-yl}propyl]-3a,7,7a-tetrahydro-1H-isoindole-1,3(2H)-dione hydrochloride; (Compound No. 27)
2-[3-{4-(2-Isopropoxyphenyl)piperazin-1-yl}propyl]-5-chloro-6-hydroxy-3a,4,5,6,7,7a-hexahydro-1H-isoindole-1,3(2H)-dione hydrochloride; (Compound No. 28)
1-[4-(2-Isopropoxyphenyl)piperazin-1-yl]-2-hydroxy-3-(2,6-dioxopiperidin-1-yl)propane hydrochloride; (Compound No. 29)
2-[3-{4-(2-Isopropoxyphenyl)piperazin-1-yl}-2-hydroxypropyl]-5,6-epoxy-3a,4,5,6,7,7a-hexahydro-1H-isoindole-1,3(2H)-dione; (Compound No. 30)
2-[3-{4-(2-(2,2,2-Trifluoroethoxyphenyl)piperazin-1-yl}-2-hydroxypropyl]-3a,4,7,7a-tetrahydro-1H-isoindole-1,3(2H)-dione hydrochloride; (Compound No. 31)
2-[3-{4-(2-(2,2,2-Trifluoroethoxy)phenyl)piperazin-1-yl}propyl]-5,6-epoxy-3a,4,5,6,7,7a-hexahydro-1H-isoindole-1,3(2H)-dione; (Compound No. 32)
2-[3-{4-(2-(2,2,2-Trifluoroethoxy)phenyl)piperazin-1-yl}propyl]-5-hydroxy-3a,4,5,6,7,7a-hexahydro-1H-isoindole-1,3(2H)-dione hydrochloride; (Compound No. 33)
2-[3-{4-(2-(2,2,2-Trifluoroethoxy)phenyl)piperazin-1-yl}-2-hydroxypropyl]-5,6-epoxy-3a,4,5,7,7a-hexahydro-1H-isoindole-1,3(2H)-dione; (Compound No. 34)
2-[3-{4-(2-Isopropoxy-3-hydroxyphenyl)piperazin-1-yl lpropyl]-3a,4,7,7a-tetrahydro-1H-isoindole-1,3(2H)-dione hydrochloride; (Compound No. 35)
1-[4-(2-Isopropoxy-5-hydroxyphenyl)piperazin-1-yl]-3-(2,6-dioxopiperdin-1-yl)piperazin-1-yl]-3-(2,6-dioxopiperidin-1-yl)propane hydrochloride; (Compound No. 36)
1-[4-(2-Isopropoxy-6-hydroxyphenyl)piperazin-1-yl]-3-(2,6-dioxopiperdin-1-yl)propane hydrochloride; (Compound No. 37)
1-[4-(2-Isopropoxy-3-hydroxyphenyl)piperazin-1-yl]-3-(2,6-dioxopiperidin-1-yl)propane hydrochloride; (Compound No. 38)
1-[4-{2-(2,2,2-Trifluoroethoxy)phenyl)piperazin-1-yl]-2-hydroxy-3-(2,6-dioxopiperidin-1-yl)propane hydrochloride; (Compound No. 39)
2-[3-{4-(2-Isopropoxyphenyl)piperazin-1-yl lpropyl]4-acetoxy-3a,4,7,7a-tetrahydro-1H-isoindole-1,3(2H)-dione hydrochloride; (Compound No. 40)
2-[3-{4-(2-Isopropoxyphenyl)piperazin-1-yl}propyl]-4-hydroxy-3a,4,7,7a-tetrahydro-1H-isoindole-1,3(2H)-dione hydrochloride; (Compound No. 41)
2-[N-{N′-(2-Isopropoxyphenyl)aminoethyl}aminopropyl]-3a,4,7,7a-tetrahydro-1H-isoindole-1,3(2H)-dione hydrochloride; (Compound No. 42)
2-[3-{4-(2-Cyclopentyloxyphenyl)piperazin-1-yl}propyl]-3a,4,7,7a-tetrahydro-1H-isoindole-1,3(2H)-dione hydrochloride; (Compound No. 43)
1-[4-(2-hydroxyphenyl)piperazin-1-yl]-2-hydroxy-3-(2,6-dioxopiperidin-1-yl]propane hydrochloride; (Compound No. 44)
2-[3-{4-(2-Biphenyl)piperazin-1-yl}propyl]-3a,4,7,7a-tetrahydro-1H-isoindole-1,3(2H)-dione hydrochloride; (Compound No. 45)
2-[N-{N′-(2-Isopropoxyphenyl)aminoethyl}acetylaminopropyl]-3a,4,7,7a-tetrahydro-1H-isoindole-1,3(2H)-dione hydrochloride; (Compound No. 46)
2-[N-{N′-(2-Isopropoxyphenyl)acetylaminoethyl}aminopropyl]-3a,4,7,7a-tetrahydro-1H-isoindole-1,3(2H)-dione hydrochloride; (Compound No. 47)
2-[N-[{N′-(2-Isopropoxyphenyl)aminoethyl}hydroxyethyl]aminopropyl]-3a,4,7,7a-tetrahydro-1H-isoindole-1,3(2H)-dione hydrochloride; (Compound No. 48)
1-[4(2-Isopropoxyphenyl)piperazin-1-yl]-1-oxo-3-(2,6-dioxopiperidin-1-yl)propane hydrochloride; (Compound No. 49)
2-[N-{N′-(2-Isopropoxyphenyl)aminoethyl}acetaldehyde-aminopropyl]-3a,4,7,7a-tetrahydro-1H-isoindole-1,3(2H)-dione; (Compound No. 50)
2-[N-{N′-(2-Isopropoxyphenyl)aminoethyl)aminopropyl-N,N′-(bis hydroxyethyl]-3a,4,7,7a-tetrahydro-1H-isoindole-1,3(2H)-dione; (Compound No. 51)
2-[N-{N′-(2-Isopropoxyphenyl)aminoethyl}ethylacetate-aminopropyl]-3a,4,7,7a-tetrahydro-1H-isoindole-1,3(2H)-dione; (Compound No. 52)
2-[N-{N′-(2-Isopropoxyphenyl)aminoethyl}formylaminopropyl]-3a,4,7,7a-tetrahydro-1H-isoindole-1,3(2H)-dione; (Compound No. 53)
2-[3-{4-(2-Isopropoxyphenyl)piperazin-3-oxo-1-yl lpropyl]-3a,4,7,7a-tetrahydro-1H-isoindole-1,3(2H)-dione; (Compound No. 54)
1-[4-(2-Methoxyphenyl)piperazin-1-yl-4-N-oxide]-3-(2,6-dioxopiperidin-1-yl]propane; (Compound No. 55)
1-[N-{N′-(2-Methoxyphenyl)aminoethyl}]-3-(2,6-dioxopiperidin-1-yl)aminopropane hydrochloride; (Compound No. 56)
1-[N—N-{N′-(2-Methoxyphenyl)aminoethyl}]-3-(2,6-dioxopiperidin-1-yl)aminopropane hydrochloride; (Compound No. 57)
2-[3-{4-(2-Isopropoxy-4-acetylaminophenyl)piperazin-1-yl}propyl]-3a,4,7,7a-tetrahydro-1H-isoindole-1,3(2H)-dione hydrochloride; (Compound No. 58)
2-[3-{4-(2-Isopropoxyphenyl)piperazin-1-yl}propyl]-7,7a-dihydro-1H-isoindole-1,3(2H)-dione hydrochloride; (Compound No. 59)
2-[3-{4-(2-Isopropoxyphenyl)piperazin-1-yl}-propyl]4-hydroxy-3a,4,5,6,7,7a-hexahydro-1H-isoindole-1,3(2H)-dione hydrochloride; (Compound No. 60)
2-[3-{4-(2-Isopropoxyphenyl)piperazin-1-yl}-propyl]-exo-4,7-epoxy-3a,4,7,7a-tetrahydro-1H-isoindole-1,3(2H)-dione hydrochloride; (Compound No. 61)
2-[3-{4-(2-Isopropoxyphenyl)piperazin-1-yl}-1-oxo-propyl]-3a,4,7,7a-tetrahydro-1H-isoindole-1,3(2H)-dione hydrochloride; (Compound No. 62)
2-[3-{4-(2-Isopropoxyphenyl)piperazin-1-yl}-1-oxo-propyl]-3a,4,5,6,7,7a-hexahydro-1H-isoindole-1,3(2H)-dione hydrochloride; (Compound No. 63)
2-[3-{4-(2-Isopropoxyphenyl)piperazin-1-yl,4-N-oxide}propyl]-3a,4,7,7a-tetrahydro-1H-isoindole-1,3(2H)-dione; (Compound No. 64)
2-[3-{4-(2-Isopropoxyphenyl)piperazin-1-yl,1-N-oxide}2-hydroxypropyl]-3a,4,7,7a-tetrahydro-1H-isoindole-1,3(2H)-dione; (Compound No. 65)
2-[3-{4-(2-Ethoxyphenyl)piperazin-1-yl}propyl]-5,6-dihydroxy-3a,4,7,7a-tetrahydro-1H-isoindole-1,3(2H)-dione hydrochloride; (Compound No. 66)
2-[3-{3-(2-Isopropoxyphenyl)imidazolidon-1-yl}propyl]-3a,4,7,7a-tetrahydro-1H-isoindole-1,3(2H)-dione; (Compound No. 67)
2-[N-{N′-(2-Isopropoxyphenyl)aminoethyl}aminopropyl-N′-(β-hydroxyethyl]-3a,4,7,7a-tetrahydro-1H-isoindole-1,3(2H)-dione hydrochloride; (Compound No. 68)
1-[4-(2-Methoxyphenyl)piperazin-1-yl-1-N-oxide]-3-(2,6-dioxopiperidin-1-yl]-2-hydroxypropane; (Compound No. 69)
1-[4-(2-Hydroxyphenyl)piperazin-1-yl-1-N-oxide]-3-(2,6-dioxopiperidin-1-yl]propane; (Compound No. 70)
2-[3-{4-(2-Isopropoxyphenyl)-2,3-dioxopiperazin-1-yl}-propyl]-3a,4,7,7a-tetrahydro-1H-isoindole-1,3(2H)-dione; (Compound No. 71)
2-[3-{4-(2-Isopropoxyphenyl)piperazin-1-yl}propyl]-4,7-dihydroxy-3a,4,7,7a-tetrahydro-1H-isoindole-1,3(2H)-dione; (Compound No. 72)
2-[3-{4-(2-Isopropoxyphenyl)piperazin-1-yl}propyl]-4,7-diacetoxy-3a,4,7,7a-tetrahydro-1H-isoindole-1,3(2H)-dione hydrochloride; (Compound No. 73)
2-[N-{N′-(2-Isopropoxyphenyl)aminoethyl}ethylaminopropyl]-3a,4,7,7a-tetrahydro-1H-isoindole-1,3(2H)-dione; (Compound No. 74)
2-[3-{4-(2-Isopropoxyphenyl)piperazin-1-yl}propyl]-5,6-dimethoxy-3a,4, 7,7a-tetrahydro-1H-isoindole-1,3 (21H)-dione hydrochloride; (Compound No. 75)
2-[3-{4-(2-Isopropoxyphenyl)piperazin-1-yl}propyl]-5,6-dimethoxy-3a,4,5,6,7,7a-hexahydro-1H-isoindole-1,3(2H)-dione hydrochloride; (Compound No. 76)
2-[3-{4-(2-Isopropoxyphenyl)piperazin-1-yl lpropyl]-4,7-diphenyl-3a,4,7,7a-tetrahydro-1H-isoindole-1,3(2H)-dione hydrochloride; (Compound No. 77)
2-[3-{4-(2-Methoxyphenyl)piperazin-1-yl}propyl]-4,7-diphenyl-3a,4,7,7a-tetrahydro-1H-isoindole-1,3(2H)-dione hydrochloride; (Compound No. 78)

Pharmaceutically acceptable, non-toxic, acid addition salts of the compounds of the present invention having the utility of the free bases of Formula I may be formed with inorganic or organic acids, by methods well known in the art and may be used in place of the free bases. Representative examples of suitable acids for formation of such acid addition salts are maleic, fumaric, benzoic, ascorbic, pamoic, succinic, bismethylene salicylic, methanesulfonic, ethane disulfonic, acetic, propionic, tartaric, salicylic, citric, gluconic, aspartic, stearic, palmitic, itaconic, glycolic, p-aminobenzoic, glutamic, benzenesulfamic, phosphoric, hydrobromic, sulfuric, cyclohexylsulfamic, hydrochloric, and nitric acids.

The present invention also includes within its scope prodrugs of the compounds of Formula I. In general, such prodrugs will be functional derivatives of these compounds which are readily converted in vivo into the defined compounds. Conventional procedures for the selection and preparation of suitable prodrugs are known.

The invention also includes the enantiomers, diastereomers, N-oxides, polymorphs, pharmaceutically acceptable salts and pharmaceutically acceptable solvates of these compounds, as well as metabolites having the same type of activity. The invention further includes pharmaceutical compositions comprising the molecules of Formula I, or prodrugs, metabolites, enantiomers, diastereomers, N-oxides, polymorphs, solvates or pharmaceutically acceptable salts thereof, in combination with a pharmaceutically acceptable carrier and optionally included excipients.

In yet another aspect, the invention is directed to methods for selectively blocking α_{1A} receptors by delivering in the environment of said receptors, e.g., to the extracellular medium (or by administering to a mammal possessing said receptors), an effective amount of the compounds of the invention.

### DETAILED DESCRIPTION OF THE INVENTION

The compounds of the present invention may be prepared by one of the reaction sequences (Schemes I-X) to yield the compounds of Formula I. The starting materials for schemes I-X may be suitably adapted to produce the more specific compounds of Formula I.

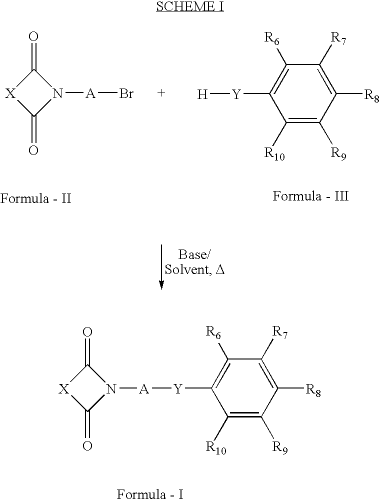

Scheme I shows the synthesis of the compounds of Formula I wherein X is selected from the group consisting of 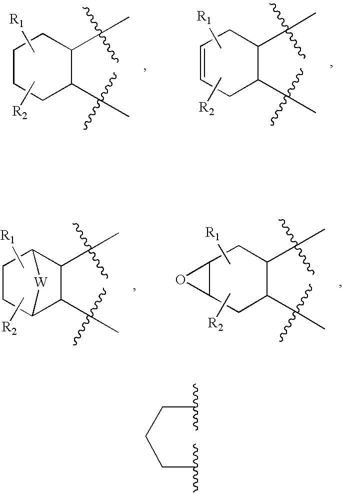
where the points of attachment are depicted by hashed bonds, and where one point of attachment is bonded to the carbonyl adjacent to the nitrogen and the second point of attachment is bonded to the other carbonyl;
W is O, S, SO or SO₂; 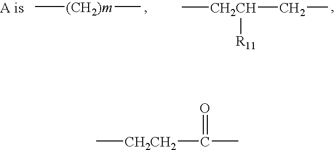
   where m is one of the integers 2, 3 or 4;
R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 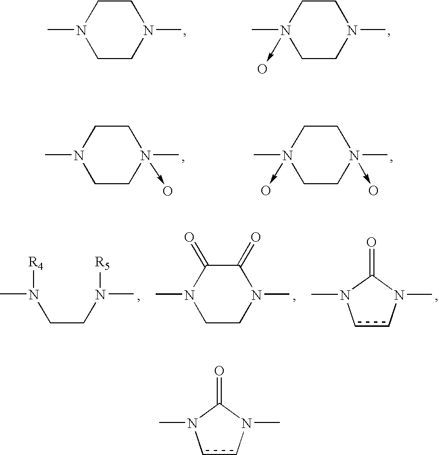
   R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₄) perhaloalkoxy lower (Cl 4) alkoxy substituted with one or more of F, Cl, Br, I, OH, or OR₃, or optionally substituted groups selected from aryl, aralalkyl, heterocyclyl or heteroaryl, said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl, optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower (C₁-C₄) alkyl amino carbonyl, hydroxy substituted with aromatic or non-aromatic five or six membered ring, phenyl or phenyl substituted by Cl, F, Br, I, NO₂, NH₂, (C₁₋₄) alkyl or (C₁₋₄) alkoxy, (C₁₋₄) perhaloalkyl, (C, ₄) perhaloalkoxy wherein the broken line 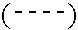
      is a single bond or no bond.

The preparation comprises condensing α,ω-dicarboximides of Formula II with substituted phenyl of Formula III, in the presence of a base and an organic solvent at a temperature ranging from about 70-150° C. for a period varying between 8-24 hours to produce the corresponding compounds of Formula I. The suitable organic solvent is a dipolar aprotic solvent selected from the group consisting of dimethylsulfoxide, N,N-dimethylformamide, hexamethylphosphoramide and N-methyl-2-pyrrolidone. The reaction is carried out in the presence of an inorganic base preferably selected from the group potassium carbonate and sodium carbonate. The preferable temperature conditions for the reaction are 70-80° C.

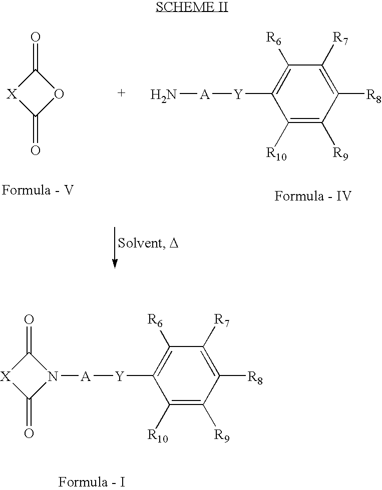

The compounds of the Formula I can also be prepared by Scheme II, wherein substituted phenyl of the Formula IV is condensed with the anhydrides of Formula V, to give compounds of Formula I, wherein X, Y, A, R₆, R₇, R₈, R₉ and R₁₀ as defined above. The reaction is carried out under reflux conditions in an organic solvent such as toluene, benzene, xylene, pyridine, acetic acid in pyridine, or mixtures thereof. The preferable temperature condition for the reaction is 70-80° C.

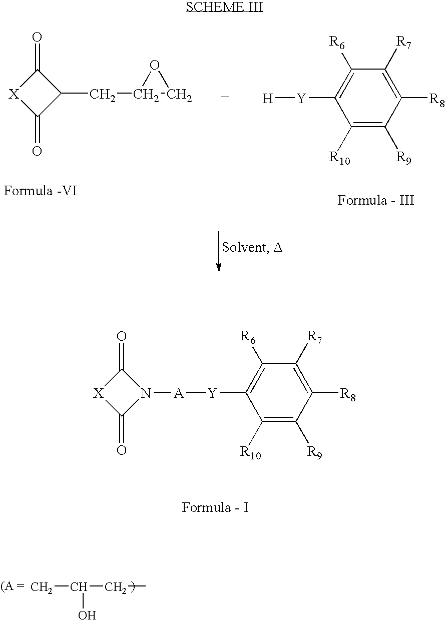

Scheme m shows the synthesis of the compounds of Formula I (when A=—CH₂—CH—CH₂) which comprises the nucleophilic ring opening of the epoxide of Formula VI with the substituted phenyl of the Formula III, wherein X, Y, R₆, R₇, R₈, R₉ and R₁₀ are as defined earlier and A is —CH₂—CH(OH)—CH₂—. Preferably, the reaction is carried out in organic solvent at a temperature ranging from 50-100° C. for a period ranging from one to several hours. The solvent for carrying out this reaction is a dipolar aprotic solvent such as dimethylsulfoxide, N,N-dimethylformamide, sulfolane, dimethylacetamide, hexamethylphosphamide and N-methyl-2-pyrrolidine. Polar protic solvents like ethanol can also be used under reflux conditions for this reaction. The reaction can be carried out in the presence of inorganic base such as potassium carbonate or sodium carbonate, or an organic base such as triethyl amine and diisopropylethylamine. The suitable temperature range for the reaction is 70-80° C.

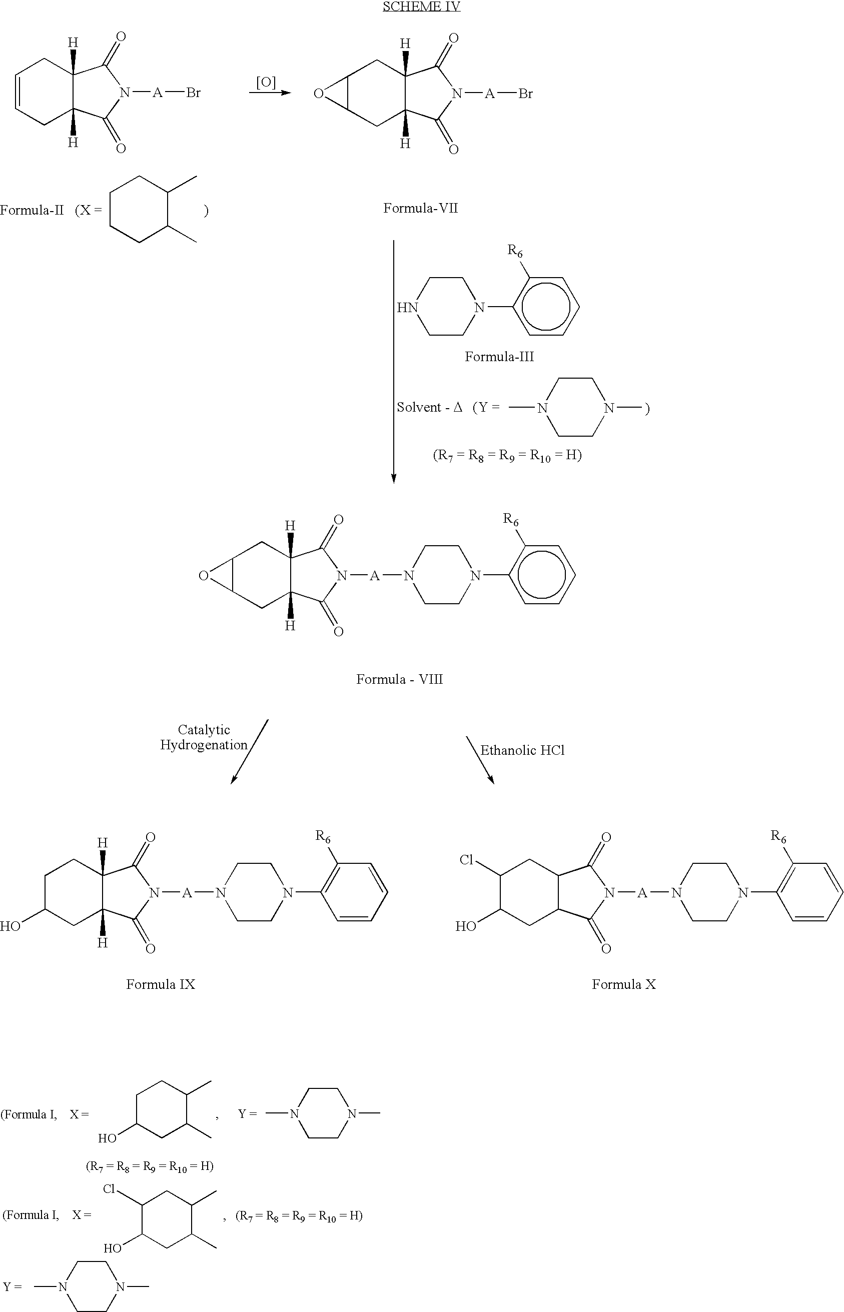

The compounds of Formula IX (Formula I, when 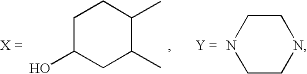
R7=R₈=R₉=R₁₀=H) can be prepared by the reaction sequence of Scheme IV, wherein A and R₆ are as defined earlier. The starting material for this scheme is the compound of Formula II (when 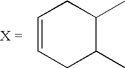
) which is subjected to epoxidation to give a compound of Formula VII wherein A is same as defined earlier. The reaction of epoxidation is carried out in a nonpolar solvent or a polar aprotic solvent at sub-zero temperatures for a period of 24-30 hours. The product (Formula VII) formed is then condensed with substituted phenyl of Formula III (when 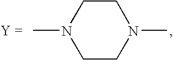
R₇=R₈=R₉=R₁₀=H) in the presence of a base and an organic solvent at a temperature ranging from 70-150° C. for a period varying between 8-24 hours to produce compound of Formula VIII. Nucleophilic ring opening of epoxide of compound of Formula VIII with alcoholic hydrochloric acid gave corresponding compound of Formula X, while catalytic hydrogenation of compounds of Formula VIII in a polar solvent at reduced pressure, for a period ranging between 36-72 hours gave corresponding compounds of Formula IX.

The epoxidation of compounds of Formula II is carried out with peracid such as metachloroperbenzoic acid, peracetic acid or trifluoroperacetic acid. The organic solvent used in this reaction can be selected from a group consisting of dichloromethane, dichloroethane, chloroform, tetrahydrofuran, acetone and acetonitrile. The preferred temperature conditions are 0-5° C. The condensation of the epoxide of Formula VII with compound of Formula III is carried out in a polar aprotic solvent such as dimethylsulfoxide, N,N-dimethylformamide, sulfolane, dimethylacetamide hexamethylphosphoramide and N-methyl-2-pyrrolidone. The inorganic base used in this reaction is selected from the group consisting of potassium carbonate and sodium carbonate and the preferable temperature for carrying out this reaction is 50-55° C. The nucleophilic epoxide ring opening of compounds of Formula VIII is carried out preferably with methanolic or ethanolic hydrochloric acid while the catalytic hydrogenation of the epoxide of compounds of Formula VIII is carried out in polar protic solvents such as methanol and ethanol.

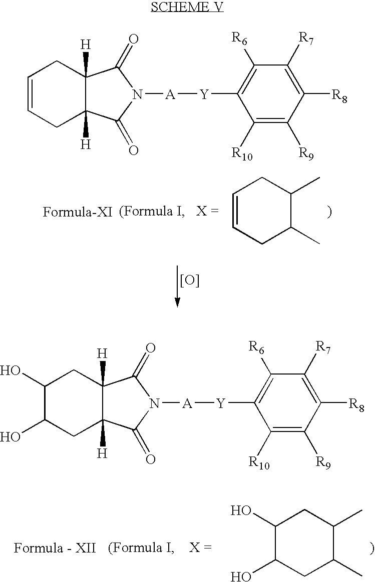

The compounds of Formula XII (Formula I when 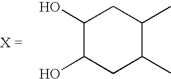
) is prepared by the method of Scheme V with Y, A, R₆, R₇, R₈, R₉, and R₁₀ groups as defined earlier. The starting material for Scheme V is the compound of Formula XI (Formula I, when 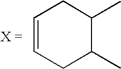
) which is subjected to oxidation to give the corresponding diol of Formula XII. The reaction is carried out preferably in a polar solvent at about 0-5° C. for about one to several hours. The oxidizing agent in this reaction is selected from the group consisting of osmium tetraoxide and potassium permanganate. The reaction is carried out in a polar protic or aprotic solvent such as methanol, ethanol, acetone, and acetonitrile. The preferable temperature range is 0-5° C.

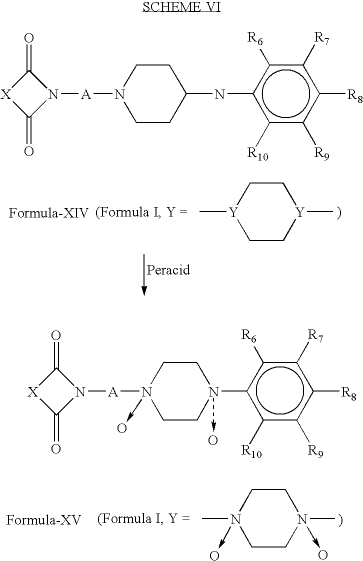

The compounds of Formula XV (Formula I, when 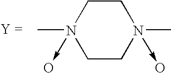
) is prepared by following the reaction sequence of Scheme VI with X, A, R₆, R₇, R₈, R₉ and R₁₀ groups as defined earlier. The starting materials for Scheme VI are compound of Formula XIV (Formula I, when 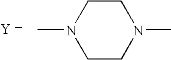
) which upon treatment with peracid such as metachloroperbenzoic acid in an organic solvent at sub zero temperature for a period varying between 2-8 hours gives the corresponding N-Oxides of Formula XV.

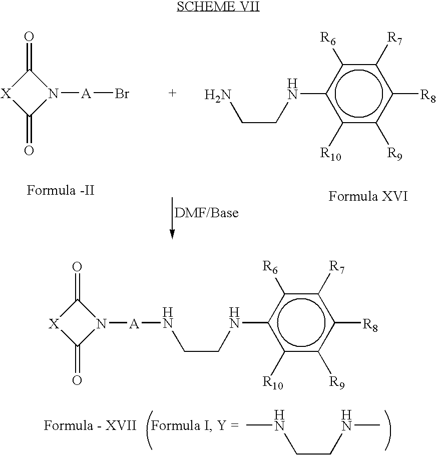

Scheme VII reveals the synthesis of the compounds of Formula XVII (Formula I, when 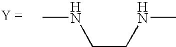
), wherein X, A, R₆, R₇, R₈, R₉ and R₁₀ are as defined earlier. The preparation comprises condensing α,ω-dicarboximides of Formula II with ethylene diamines of Formula XVI in the presence of a base and an organic solvent at a temperature ranging from 70-80° C. for a period varying between 8-24 hours to produce the corresponding compounds of Formula XVII.

The suitable organic solvent is a dipolar aprotic solvent, which is selected from the group consisting of dimethyl sulfoxide, N,N-dimethylformamide, sulfolane, dimethyl acetamide, hexamethyl phosphoramide and N-methyl-2-pyrrolidone. The reaction is carried out in the presence of an inorganic base, preferably selected from the group consisting of potassium carbonate and sodium carbonate. The preferable temperature conditions for the reaction are 70-80° C.

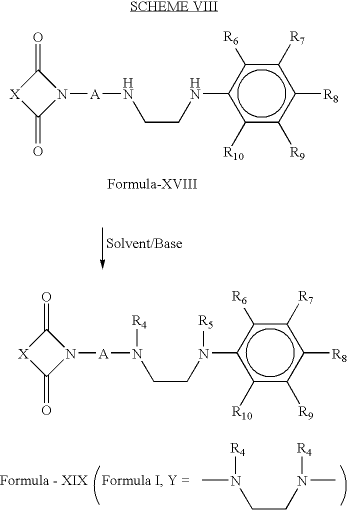

The compounds of Formula XVM are alkylated in, the presence of an inorganic base and organic solvent at a temperature ranging between 20-150° C. for a period varying between 5-24 hours to give the compounds of Formula XIX (Formula I, when 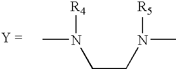
) with X, A, R₄, R₅, R₆, R₇, R₈, R₉ and R₁₀ are the same as defined earlier.

The suitable organic solvent is a dipolar aprotic solvent which is selected from the group consisting of dimethylsulfoxide, N,N-dimethylformamide, sulfolane, dimethylacetamides, hexamethyl phosphoramide and N-methyl-2-pyrrolidone. The reaction is carried out in the presence of an inorganic base, preferably selected from the group consisting of potassium carbonate, sodium carbonate and sodium hydride. The preferable temperature conditions for the reaction are 120-150° C.

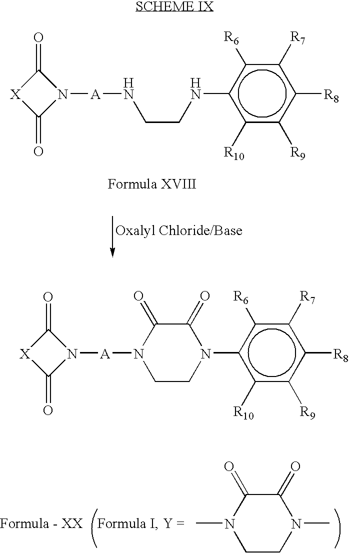

The compounds of Formula XVIII are treated with oxalyl chloride in the presence of an organic base and organic solvent at temperature ranging between 0-20° C. for a period varying between 1-5 hours which yields the corresponding dioxopiperazine of Formula XX (Formula I, when 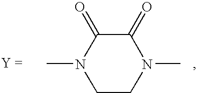
with X, A, R₆, R₇, R₈, R₉ and R₁₀ are the same as defined earlier.

The suitable organic solvent is selected from the group consisting of dichloromethane, dichloroethane, chloroform and tetrahydrofuran. The reaction is carried out in the presence of an organic base preferably selected from the group triethylamine and diisopropyl ethylamine.

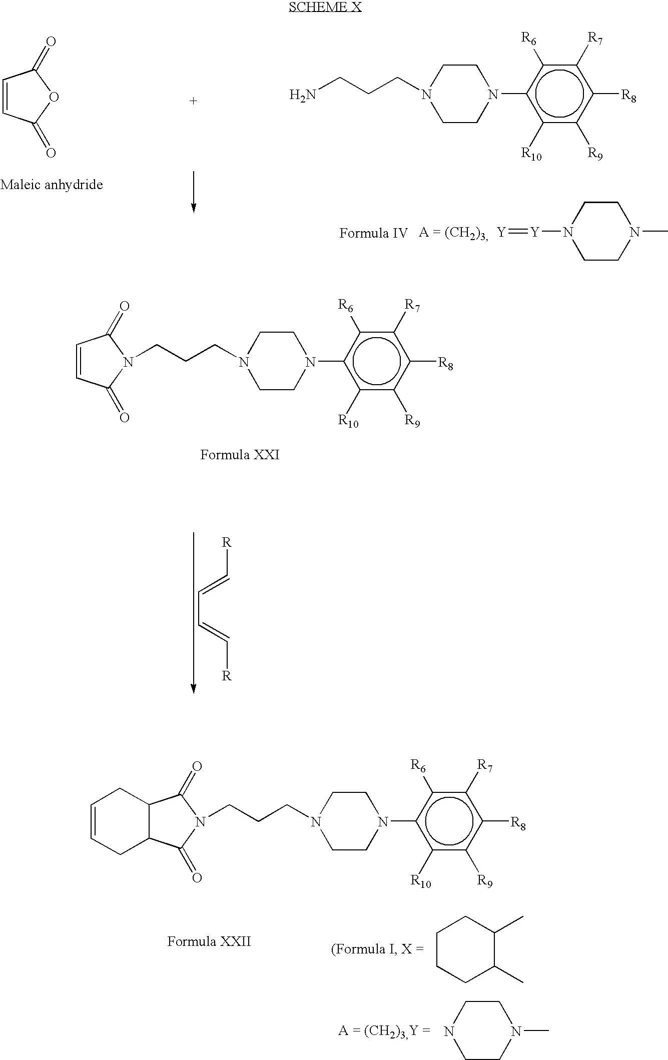

Scheme X shows the synthesis of the compounds of Formula XXIII (Formula I, when 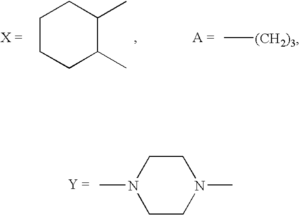
) in which R₆, R₇, R₈, R₉ and R₁₀ are as defined earlier which comprises condensing maleic anhydride with substituted phenyl piperazine of Formula IV 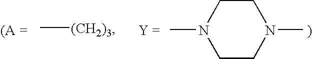
in an organic solvent under reflux condition with azeotropic removal of water to give the corresponding α,ω-dicarboximide of Formula XXI which is further subjected to Diels Alder addition with substituted butadienes in a non-polar organic solvent under reflux conditions to give the corresponding compounds of Formula XXII. The non-polar organic solvent for carrying out this reaction is chosen from the group consisting of toluene, benzene and xylene. The preferable temperature conditions are 70-80° C.

The examples mentioned below demonstrate the general synthetic procedure as well as the specific preparation of the preferred compounds. The examples are given to illustrate the details of the invention and should not be construed to limit the scope of the present invention.

### EXAMPLE 1

### Scheme I

### Preparation of 2-[3-{4-(4-(2-(2,2,2-Trifluoroethoxy)phenyl)piperazin-1-yl}propyl]-3a,4,7,7a-tetrahydro-1-H-isoindole-1,3-(2H)-dione hydrochloride;

A mixture of 1-(3-bromopropyl)-cis-3a,4,7,7a-tetrahydrophthalimide (1 g, 3.67 mmol), 1-(2-(2,2,2-trifluoroethoxy)phenyl)piperazine (1.43 g, 5.5 mmol) and potassium iodide (0.036 g, 0.22 mmol) in NN-dimethyl formamide (25 ml) was heated at 70-80° C. for about 12 hours. After the reaction was over, solvent was evaporated under reduced pressure, the residue was suspended in water (100 ml) and extracted with ethyl acetate (2×50 ml). The combined ethyl acetate layer was washed with water (2×50 ml), dried over anhydrous sodium sulphate, and the solvent evaporated in vacuo to yield crude oil. The product was purified by chromatography on silica gel, using dichloromethane/methanol (98/2, v/v) as eluent to afford the suitable compound 1 g as an oil. The compound so obtained was converted in to its hydrochloride salt as off-white solid (m.p. 204-208° C.).

MS: m/z 452.3 (MH⁺), IR (KBr cm⁻¹): 1697.7 (C—O) ¹HNMR (DMSO-d₆) δ: 1.92 (2H, m), 2.23-2.39 (4H, dd), 3.05-3.19 (8H, m), 3.43-3.55 (6H, m), 4.69-4.73 (2H, q), 5.89 (2H, s), 7.03-7.06 (4H, m).

An illustrative list of the compounds of the invention which were synthesised by the above method is given below:
1-[4-(2-Hydroxyphenyl)piperazin-1-yl]-3-(2,6-dioxopiperidin-1-yl)propane hydrochloride; mp 224-227° C.
1-[4-{2-(2,2,2-Trifluoroethoxy)phenyl piperazin-1-yl]-3-(2,6-dioxopiperidin-1-yl)propane-hydrochloride; m.p. 208-212° C.
1-[4-(2-Ethoxyphenyl)piperazin-1-yl]-3-(2,6-dioxopiperadin-1-yl)ethane hydrochloride; m.p. 199-202° C.
2-[2-{4-(2-Ethoxyphenyl)piperazin-1-yl}ethyl]-3a,4,7,7a-tetrahydro-1H-isoindole-1,3(2H)-dione hydrochloride, m.p. 220-222° C.
2-[2-{4-(2-(2,2,2-Trifluoroethoxy)phenyl)piperazin-1-yl}ethyl]-3a,4,7,7a-tetrahydro-1H-isoindole-1,3(2H)-dione hydrochloride; m.p. 178-180° C.
2-[3-{4-(2-Isopropoxy-5-hydroxyphenyl)piperazin-1-yl}propyl]-3a,4,7,7a-tetrahydro-1H-isoindole-1,3(2H)-dione hydrochloride; m.p. 238-242° C.
2-[3-{4-(2-Ethoxy-5-hydroxyphenyl)piperazin-1-yl}propyl]-3a,4,7,7a-tetrahydro-1H-isoindole-1,3-(2H)-dione hydrochloride; m.p. 234-236° C.
2-[3-{4-(2-Isopropoxy-4-nitrophenyl)piperazin-1-yl lpropyl]-3a,4,7,7a-tetrahydro-1H-isoindole-1,3(2H)-dione hydrochloride; m.p. 199-203° C.
2-[3-{4-(2-Isopropoxy-4-aminophenyl)piperazin-1-yl}propyl]-3a,4,7,7a-tetrahydro-1H-isoindole-1,3(2H)-dione hydrochloride; m.p. 220-222° C.
2-[3-{4-(2-isopropoxy-6-hydroxyphenyl)piperazin-1-yl}propyl]-3a,4,7,7a-tetrahydro-1H-isoindole-1,3(2H)-dione hydrochloride; m.p. 217-220° C.
2-[3-{4-(2-isopropoxy-3-hydroxyphenyl)piperazin-1-yl}propyl]-3a,4,7,7a-tetrahydro-1H-isoindole-1,3(2H)-dione hydrochloride; m.p. 212-216° C.
1-[4-(2-Isopropoxy-5-hydroxyphenyl)piperazin-1-yl]-3-(2,6-dioxopiperdin-1-yl)piperazin-1-yl]-3-(2,6-dioxopiperidin-1-yl)propane hydrochloride; m.p. 218-222° C.
1-[4-(2-Isopropoxy-6-hydroxyphenyl)piperazin-1-yl]-3-(2,6-dioxopiperdin-1-yl)propane hydrochloride; m.p. 215-219° C.
1-[4-(2-Isopropoxy-3-hydroxyphenyl)piperazin-1-yl]-3-(2,6-dioxopiperidin-1-yl)propane hydrochloride; m.p. 260-263° C.
2-[3-{4-(2-Cyclopentyloxyphenyl)piperazin-1-yl}propyl]-3a,4,7,7a-tetrahydro-1H-isoindole-1,3(2H)-dione hydrochloride; mp 185-189° C.
2-[3-{4-(2-Biphenyl)piperazin-1-yl}propyl]-3a,4,7,7a-tetrahydro-1H-isoindole-1,3(2H)-dione hydrochloride; mp 164-168° C.
1-[4-(2-Isopropoxyphenyl)piperazin-1-yl]-1-oxo-3-(2,6-dioxopiperidin-1-yl)propane hydrochloride; m.p. 174-177° C.
2-[3-{4-(2-Isopropoxy-4-acetylaminophenyl)piperazin-1-yl}propyl]-3a,4,7,7a-tetrahydro-1H-isoindole-1,3(2H)-dione hydrochloride; mp 226-228° C.
2-[3-{4-(2-Isopropoxyphenyl)piperazin-1-yl}-1-oxo-propyl]-3a,4,7,7a-tetrahydro-1H-isoindole-1,3(2H)-dione hydrochloride; m.p 220-222° C.
2-[3-{4-(2-Isopropoxyphenyl)piperazin-1-yl}-1-oxo-propyl]-3a,4,5,6,7,7a-hexahydro-1H-isoindole-1,3(2H)-dione hydrochloride; m.p. 227-229° C.

### EXAMPLE 2

### Scheme III

### Preparation of 2-[3-{4-(2-isopropoxyphenyl)piperazin-1-yl}-2-hydroxypropyl]-3a,4,7,7a-tetrahydro-1H-isoindole-1,3(2H)-dione hydrochloride;

A mixture of 1-(2,3-epoxypropyl)-cis-3a,4,7,7a-tetrahydrophthalimide (0.5 g, 2.42 mmol), 1-(2-isopropoxyphenyl)piperazine (0.48 g, 2.18 mmol) and triethylamine (0.27 g, 2.67 mmol) in ethanol (35 ml) were refluxed for 5 hours. After the reaction was over, the solvent was removed under reduced pressure The residue thus obtained was suspended in water (50 ml), and extracted with dichloromethane (2×50 ml). The combined dichloromethane layer was washed with water (50 ml), dried over anhydrous sodium sulphate, and finally concentrated to yield a crude oil. The product was purified by chromatography on silica gel, using chloroform/methanol (98/2, v/v) to afford the product 0.8 g (77.7%) as an oil.

The hydrochloride salt was prepared by the addition of equimolar quantity of ethereal hydrochloride to the ethanolic solution of free base. The solid was precipitated by the addition of diethylether and collected by filtration. m.p. 206-209° C.

MS: m/z 429 (ME) IR (KBr cm¹) 3369.3 (—OH), 1695 (C=0) ¹HNMR (CDCl₃) δ: 1.38-1.40 (6H, d), 2.19-2.26 (2H, dd), 2.57-2.63 (2H, dd), 3.09-3.24 (5H, m), 3.52-3.58 (4H, m), 3.65-3.69 (4H, m), 3.72-3.76 (1H, d), 4.58-4.64 (2H, m), 5.89-5.91 (2H, m), 6.88-6.93 (2H, m), 7.05-7.10 (2H, m).

An illustrative list of the compounds of the invention which were synthesised by the above method is given below:
2-[3-{4-(2-Methoxyphenyl)piperazin-1-yl}-2-hydroxypropyl]-3a,4,7,7a-tetrahydro-1H-isoindole-1,3(2H)-dione hydrochloride; m.p. 205-207° C.,
2-[3-{4-(2-Ethoxyphenyl)piperazin-1-yl}-2-hydroxypropyl]-3a,4,7,7a-tetrahydro-1H-isoindole-1,3-(2H)-dione hydrochloride; m.p. 224-226° C.,
2-[3-{4-(2-Hydroxyphenyl)piperazin-1-yl}-2-hydroxypropyl]-3a,4,7,7a-tetrahydro-1H-isoindole-1,3(2H)-dione hydrochloride; m.p. 258-260° C.,
1-[4-(2-Isopropoxyphenyl)piperazin-1-yl]-2-hydroxy-3-(2,6-dioxopiperidin-1-yl)propane hydrochloride; m.p. 180-183° C.,
2-[3-{4-(2-Isopropoxyphenyl)piperazin-1-yl}-2-hydroxypropyl]-5,6-epoxy-3a,4,5,6,7,7a-hexahydro-1H-isoindole-1,3(2H)-dione; m.p. obtained as oil.
2-[3-{4-(2-(2,2,2-Trifluoroethoxyphenyl)piperazin-1-yl}-2-hydroxypropyl]-3a,4,7,7a-tetrahydro-1H-isoindole-1,3(2H)-dione hydrochloride; m.p. 183-186° C.,
2-[3-{4-(2-(2,2,2-Trifluoroethoxy)phenyl)piperazin-1-yl}-2-hydroxypropyl)-5,6-epoxy-3a,4,5,7,7a-hexahydro-1H-isoindole-1,3(2H)-dione; m.p. oil.
1-[4-{2-(2,2,2-Trifluoroethoxy)phenyl)piperazin-1-yl]-2-hydroxy-3-(2,6-dioxopiperidin-1-yl)propane hydrochloride; m.p. 146-150° C.,
1-[4-(2-hydroxyphenyl)piperazin-1-yl]-2-hydroxy-3-(2,6-dioxopiperidin-1-yl]propane hydrochloride; m.p. 202-207° C.

### EXAMPLE 3

### Scheme II

### Preparation of 2-[3-{4-(2-isopropoxyphenyl)piperazin-1-yl}propyl)piperazin-1-yl}propyl]-4-hydroxy-3a,4,7,7a-tetrahydro-1H-isoindole-1,3(2H)-dione hydrochloride

### Example 3A

### Preparation of 2-[3-{4-(2-Isopropoxyphenyl)piperazin-1-yl}propyl]-4-acetoxy-3a,4,7,7a-tetrahydro-1H-isoindole-1,3(2H)-dione hydrochloride;

A mixture of 1-amino-3-[4-(2-isopropoxyphenyl)piperazin-1-yl]propane (1.19 g, 4.3 mmol) and 3-acetoxy-1,2,3,6-tetrahydrophthalic anhydride (1 g, 4.77 mmol) in toluene (10 ml) was refluxed for 3 hours. After the reaction was over, solvent was evaporated under reduced pressure and the residue was dissolved in ethyl acetate (20 ml). The ethyl acetate solution was washed with water (2×10 ml), dried over anhydrous sodium sulphate, and concentrated in vacuo to yield crude oil. The product was purified by chromatography on silica gel, using dichloromethane/methanol (98/2, v/v) as eluent to afford 1.2 g product as a yellowish oil Yield: 59.7%. The compound so obtained was onverted in to its hydrochloride salt (m.pt. 224-227° C.).

MS: m/z 470 (MH⁺) IR (KBr cm⁻¹) 1699.6 (CO) ¹HMR (CDCl₃) δ: 1.36-1.38 (6H, d), 2.08 (3H, s), 2.22-2.25 (3H, m), 2.66 (1H, m), 3.01-3.02 (4H, m), 3.25-3.27 (1H, m), 3.52-3.65 (9H, m), 4.58-4.60 (1H, m), 5.39-5.42 (1H, m), 6.05-6.06 (2H, m), 6.86-6.92 (3H, m), 7.00-7.03 (1H, m), 12.75 (1H, br s).

### Example 3B

### Preparation of 2-[3-{4-(2-isopropoxyphenyl)piperazin-1-yl}propyl)piperazin-1-yl}propyl]-4-hydroxy-3a,4,7,7a-tetrahydro-1H-isoindole-1,3(2H)-dione hydrochloride

The product of Example 3A (compound 49) (0.7 g, 1.38 mmol) was dissolved in 1N methanolic hydrochloride (5 ml) and stirred for 3 hours at room temperature. After the reaction was over, the pH of the reaction mixture was adjusted to 7, using sodium bicarbonate solution (5% w/v), and extracted with dichloromethane (2×20 ml). The combined dichloromethane layer was washed with water (10 ml), dried over anhydrous sodium sulphate, and concentrated in vacuo to yield the crude product as an oil. The product thus obtained was purified using dichloromethane/methanol (98/2 v/v) as eluent to afford 0.51 g of the product as oil. Yield: 86.3%. The product thus obtained was converted in to its hydrochloride salt (m.pt. 186-190° C.).

MS: m/z 428 (1) ¹H NMR (CDCl₃) δ: 1.35-1.37 (6H, d), 2.37-2.47 (3H, m), 2.78-2.84 (1H, d), 3.07-3.12 (6H, m), 3.50-3.59 (6H, m), 3.64-3.68 (2H, m), 4.58-4.63 (2H, m), 5.97-5.60 (1H, m), 6.13-6.14 (1H, m) 6.13-6.14 (1H, m), 6.86-6.95 (3H, m), 7.01-7.04 (1H, m), 12.12 (1H, brs)

An illustrative list of the compounds of the invention which were synthesised by the above method is given below:
2-[3-{4-(2-Isopropoxyphenyl)piperazin-1-yl}propyl]-4,7-dihydroxy-3a,4,7,7a-tetrahydro-1H-isoindole-1,3(2H)-dione hydrochloride, m.p. 208-210° C.,
2-[3-{4-(2-Isopropoxyphenyl)piperazin-1-yl}-propyl]-exo-4,7-epoxy-3a,4,7,7a-tetrahydro-1H-isoindole-1,3(2H)-dione hydrochloride; m.p. 194-196° C.,
2-[3-{4-(2-Isopropoxyphenyl)piperazin-1-yl}-propyl]4,7-dihydroxy-3a,4,7,7a-tetrahydro-1H-isoindole-1,3(2H)-dione hydrochloride; m.p. 208-210° C.

### EXAMPLE 4

### Scheme IV

### Preparation of 2-[3-{4-(2-isopropoxyphenyl)piperazin-1-yl}propyl]-5-hydroxy-3a,4,5,6,7,7a-hexahydro-1H-isoindole-1,3(2H)-dione hydrochloride

### Example 4A

### Preparation of 2-(3-Bromopropyl)-5,6-epoxy-as-3a, 4,5,6,7,7a-hexahydro-1H-isoindole-1,3(2H)-dione (intermediate)

2-(2-Bromopropyl)-cis-3a, 4,7,7a-tetrahydro-1H-isoindole-1,3(2H)-dione (ref. 6 g, 220 mmol) was dissolved in dichloromethane (50 ml) and cooled to 0° C. A solution of m-chloroperbenzoic acid (3.8 g, 220 mmol) in dichloromethane (25 ml) was then added slowly over a period of 15 minutes to the above solution at 0-5° C. The reaction mixture was further stirred for 24 hours at the same temperature. After the reaction was over, the reaction mixture was poured in to a stirred aqueous potassium carbonate solution (2.5%, 200 ml). The resulting mixture was extracted with dichloromethane (2×100 ml). The combined organic layer was dried over anhydrous sodium sulphate. The solvent was removed under reduced pressure and the crude product thus obtained was crystallised with ethyl acetate-hexane to afford 5 g (79%) of the required intermediate which was used as such in the next step.

### Example 4B

### Preparation of 2-[3-{4-isopropoxyphenyl)piperazin-1-yl}propyl]-5,6-epoxy 3a,4,5,6,7,7a-hexahydro-1H-isoindole-1,3(2H)-dione

The intermediate compound resulting from Example 4A (4.93 g, 17.1 mmol) was dissolved in dimethylformamide (25 ml). To this solution, 1-(2-isopropoxyphenyl) piperazine hydrochloride (4 g, 15.5 mmol) was added followed by anhydrous potassium carbonate (4.29 g, 31 mmol). The reaction mixture was heated at 50° C. for about 16 hours. After the reaction was over, the solvent was removed under reduced pressure and the residue thus obtained was suspended in cold water (100 ml) and extracted with ethyl acetate (2×100 ml). The combined ethyl acetate layer was washed with water (2×100 ml) and dried over anhydrous sodium sulphate. The organic layer was concentrated in vacuo and purified by chromatography on silica gel using 4% methanol in dichloromethane as eluent to yield the title compound as an oil.

Yield 6 g (90%) MS m/z: 427.9 (MH⁺) IR (DCM cm⁻¹): 1698.7 (C═O) ¹H NMR (300 Mz, CDCl₃) δ: 1.33 (6H, d), 0.81-1.86 (2H, m); 2.13-2.20 (2H, m), 2.46 (2H, t), 2.6 (4H, s), 2.70-2.75 (4H, m), 3.09-3.15 (6H, m), 3.59 (2H, t), 4.57-4.61 (1H, m), 6.83-6.92 (4H, m)

### Example 4C

### Preparation of 2-[3-{4-(2-Isopropoxyphenyl)piperazine-1-yl}propyl]-5-hydroxy-3a,4,5,6,7,7a-hexahydro-1H-isoindole-1,3(2H)-dione hydrochloride

The compound resulting from Example 4B (0.5 g, 1.17 mmol) was dissolved in methanol (25 ml) and 10% Pd/c (0.5 g) was added. The reaction mixture was hydrogenated at 70 psi for 36 hours. After the reaction was over, the catalyst was filtered, washed with methanol (10 ml) and the solvent was evaporated. Water (50 ml) was added to the residue and extracted with dichloromethane (2×50 ml). The combined organic layer was washed with water (50 ml), dried over anhydrous sodium sulphate and concentrated. The product was purified by chromatography on silica gel using 5% methanol in dichloromethane as eluent to afford the product as an oil. Yield 0.2 g Yield: 39.8%. The hydrochloride salt was prepared by the addition of molar quantity of ethereal hydrochloride to the ethanolic solution of free base and the obtained solid was collected by filtration m.pt 213-216° C.

MS m/Z: 430 (MH⁺) IR (KBr cm⁻¹):1698 (C═O) ¹H NMR (300 MHz, CDCl₃) δ: 1.43 (6H, d), 1.79-1.83 (4H, m), 2.06-2.37 (4H, m), 2.91 (2H, bs), 3.11-3.94 (12H, m), 4.19 (1H, bs), 4.64-4.68 (1H, m), 6.92-7.16 (4H, m).

An illustrative list of the compounds of the invention which were synthesised by the above method is given below:
2-[3-{4-(2-Ethoxyphenyl)piperazin-1-yl}propyl)-5-chloro-6-hydroxy-3a,4,5,6,7,7a-hexahydro-1H-isoindole-1,3(2H)-dione hydrochloride; m.p. 190-194° C.,
2-[3-{4-(2-Ethoxyphenyl)piperazin-1-yl}propyl]-5-hydroxy-3a,4,5,6,7,7a-hexahydro-1H-isoindole-1,3 (2H)-dione hydrochloride; m.p. 210-213° C.,
2-[3-{4-(2-Isopropoxyphenyl)piperazin-1-yl}propyl]-5-chloro-6-hydroxy-3a,4,5,6,7,7a-hexahydro-1H-isoindole-1,3(2H)-dione hydrochloride; m.p. 160-164° C.,
2-[3-{4-(2-(2,2,2-Trifluoroethoxy)phenyl)piperazin-1-yl}propyl]-5,6-epoxy-3a,4,5,6,7,7a-hexahydro-1H-isoindole-1,3(2H)-dione; m.p. oil,
2-[3-{4-(2-(2,2,2-Trifluoroethoxy)phenyl)piperazin-1-yl}propyl]-5-hydroxy-3a,4,5,6,7,7a-hexahydro-1H-isoindole-1,3(2H)-dione hydrochloride; m.p. 183-186° C.

### EXAMPLE 5

### Scheme V

### Preparation of 2-[3-{4-(2-Isopropoxyphenyl)piperazin-1-yl}propyl]-5,6-dihydroxy-3a,4,5,6,7,7a-hexhydro-1H-isoindole-1,3 (211)-dione hydrochloride

2-[3-{4-(2-Isopropoxyphenyl)piperazin-1-yl}propyl]-3a,4,7,7a-tetrahydro-1H-isoindole-1,3(2H)-dione hydrochloride (1.8 g, 4 mmol), prepared using the procedure described in Example-1, was dissolved in ethanol (36 ml) and cooled to 0-5° C.

Aqueous sodium hydroxide solution (0.16 g in 5 ml, 4 mmol) was added followed by addition of aqueous solution of potassium permanganate (0.76 g, 4.8 mmol) at 0-5° C. and stirred for 4 hours at the same temperature. After the reaction was over, the precipitated magariese dioxide was filtered, washed with dichloromethane (25 ml). The solvent was removed under reduced pressure, water (50 ml) was added and extracted with dichloromethane (2×50 ml). The organic phase was dried over anhydrous sodium sulphate, concentrated in vacuo and the residue thus obtained was purified by chromatography on silica gel using 10% methanol in dichloromethane as eluent to afford 0.55 g (30.7%) of the product. The hydrochloride salt of the title compound was prepared in quantitative yield by the addition of molar quantity of ethanolic hydrogen chloride solution to a ethanolic solution of free base and the resultant precipitate was collected by filtration;

m.p. 213-216° C. MS m/z: 446.3 (MH⁺) IRKBrcm-1: 1693.4 (x=0) ¹HNMR (300 M}z, DMSO-d₆) δ:1.27 (6H, d), 1.66-1.70 (2H, m), 1.89-1.93 (4H, m), 2.93-3.16 (8H, m), 3.36-3.50 (8H, m), 4.57-4.65 (1H, m), 6.83-6.98 (4H, m)

An illustrative list of the compounds of the invention which were synthesised by the above method is given below:
2-[3-{4-(2-Ethoxyphenyl)piperazin-1-yl}propyl]-5,6-dihydroxy-3a,4,5,6,7,7a-hexahydro-1H-isoindole-1,3(2H)-dione; m.p. low melting semisolid,
2-[3-{4-(2-Isopropoxyphenyl)piperazin-1-yl}propyl]-5,6-dihydroxy-3a,4,5,6,7,7a-hexahydro-1H-isoindole-1,3(2H)-dione; m.p. low melting semisolid,
2-[3-{4-(2-Ethoxyphenyl)piperazin-1-yl}propyl)-5,6-dihydroxy-3a,4,7,7a-tetrahydro-1H-isoindole-1,3(2H)-dione hydrochloride; m.p. 222-225° C.,

### EXAMPLE 6

### Scheme VI

### Preparation of 2-[3-{4-(2-Ethoxyphenyl)piperazin-1-yl,1-N-oxide}propyl]-3a,4,7,7a-tetrahydro-1H-isoindole-1,3(2H)-dione

2-[3-{4-(2-Ethoxyphenyl)piperazin-1-yl}propyl]-3a,4,7,7a-tetrahydro-1H-isoindole-1,3 (2H)-dione (0.5 g, 1.26 mmol), prepared by the method as described in Example-I, was dissolved in dichloromethane (10 ml) and cooled to 0° C. A solution of m-chloroperbenzoic acid (0.217 g, 1.26 mmol) in dichloromethane (5 ml) was then added slowly over a period of 10 minutes, and the reaction mixture was further stirred for 2 hours at 0-5° C. and then left at room temperature overnight. After the reaction was over, it was poured into aqueous potassium carbonate solution (5%, 30 ml). The organic layer was separated, dried over sodium sulphate, and concentrated. The crude product was purified by chromatography on silica gel, using 10% methanol in dichloromethane as eluent to afford the title compound Yield 0.11 g (21%) m.p. 75-80° C.,

IRKBrcm⁻¹: 1694 (c=0) MS m/z: 414 (MH⁺) ¹HNMR (300 MHz, CDCl₃) δ: 1.44 (3H, t), 2.24-2.65 (6H, m), 3.11 (2H, t), 3.22-3.23 (4H, m), 3.29-3.44 (4H, m), 3.62-3.66 (4H, m), 4.06-4.09 (2H, q), 5.90-5.92 (2H, m), 6.85-7.02 (4H, m).

An illustrative list of the compounds of the invention which were synthesised by the above method is given below:
2-[3-{4-(2-Isopropoxyphenyl)piperazin-1-yl, 1-N-oxide}propyl]-3a,4,7,7a-tetrahydro-1H-isoindole-1,3(2H)-dione; m.p. 85-89° C.,
2-[3-{4-(2-Isopropoxyphenyl)piperazin-1-yl,1,4-N,N-dioxide}propyl]-3a,4,7,7a-tetrahydro-1H-isoindole-1,3-(2H)-dione; m.p. 178-180° C.,
2-[3-{-(2-Ethoxyphenyl)piperazin-1-yl,1,4N,N-dioxide}propyl]-3a-4,7,7a-tetrahydro-1H-isoindole-1,3(2H)-dione; m.p. 176-178° C.,
2-[3-{4-(2-Hydroxyphenyl)piperazin-1-yl, 1-N-oxide}propyl]-3a,4,7,7a-tetrahydro-1H-isoindole-1,3(2H)-dione; m.p. 198-202° C.,
1-[4-(2-Methoxyphenyl)piperazin-1-yl-4-N-oxide]-3-(2,6-dioxopiperidin-1-yl]propane; m.p. 190-194° C.,
2-[3-{4-(2-Isopropoxyphenyl)piperazin-1-yl, 1-N-oxide}propyl]-3a,4,7,7a-tetrahydro-1H-isoindole-1,3(2H)-dione,
2-[3-{4-(2-Isopropoxyphenyl)piperazin-1-yl, 1-N-oxide}propyl]-2-hydroxypropyl]-3a,4,7,7a-tetrahydro-1H-isoindole-1,3(2H)-dione hydrochloride; m.p. 191-197° C.,
1-[4-(2-Methoxyphenyl)piperazin-1-yl,1-N-oxide]-3-(2,6-dioxopiperidin-1-yl]-2-hydroxypropane; m.p. 178-182 C.,
1-[4-(2-Hydroxyphenyl)piperazin-1-yl,1-N-oxide]-3-(2,6-dioxopiperidin-1-yl]propane; m.p. 186-190° C.

### EXAMPLE 7

### Scheme VII

### Preparation of 2-[[N-{N′-(2-Isopropoxyphenyl)aminoethyl}aminopropyl]-3a,4,7,7a-tetrahydro-1H-isoindole-1,3(2H)-dione hydrochloride

A mixture of 1-(3-bromopropyl)-cis-3a, 4,7,7a-tetrahydrophthalimide (6.0 g, 22 mmol), N-(β-aminoethyl)-o-isopropoxy aniline (4.27 g, 22 mmol) and potassium carbonate (3.0 g, 22 mmol) in N,N-dimethyl formamide (30 ml) was stirred at 3040° C. for about 24 hrs. After the reaction was over, reaction mixture was poured in cold water (300 ml) and extracted with ethyl acetate (2×100 ml). The combined ethyl acetate layer was washed with water (2×100 ml), dried over anhydrous sodium sulphate, and concentrated in vacuo to yield crude oil. The crude product was purified by column chromatography on silica gel, using dichloromethane/methanol (9/1, v/v) as eluent to afford the desired compound as an oil. The compound thus obtained was converted into its hydrochloride salt as off white solid m.p. 168-170° C.

Yield 5.5 g (64%) MS m/z 386.5 (MH⁺), IR KBr cm⁻¹ 1702.9 (C═O). ¹H NMR (CDCl₃) δ: 1.37-1.39 (6H, d), 2.14-2.19 (4H, m), 2.53-2.58 (2H, b s), 3.11 (2H, b s), 3.25 (2H, b s), 3.47-3.49 (2H, m), 3.76 (2H, m), 4.53-4.62 (1H, m), 5.85-5.89 (2H, m), 6.83-6.95 (4H, m).

An illustrative list of the compounds of the invention which were synthesised by the above method is given below:
1[N-(β-aminoethyl)-2-methoxyaniline]-3-(2,6-dioxopiperidin-1-yl]propane hydrochloride; m.p. 198-201° C.

### EXAMPLE 8

### Scheme VIII

### Preparation of 2-[[N-{N′-(2-Isopropoxyphenyl)aminoethyl}hydroxyethyl]aminopropyl]-3a,4,7,7a-tetrahydro-1H-isoindole-1,3(2H)-dione hydrochloride;

The compound resulting from Example 7 (1 g, 2.6 mmol) was dissolved in N,N-dimethylformamide (10 ml). To this solution chloroethanol (0.209 g, 2.6 mmol) was added, followed by anhydrous potassium carbonate (0.36 g, 2.6 mmol). The reaction mixture was heated to 120-124° C. for 4 hrs. After the reaction was over, reaction mixture was poured into cold water (100 ml) and extracted with ethyl acetate (2×100 ml). The combined ethyl acetate layer was washed with water (2×100 ml) and dried over anhydrous sodium sulphate. The organic phase was concentrated in vacuo and purified by column chromatography on silica gel, using dichloromethane/methanol (90/10, v/v) as eluent to give the desired compound as an oil. The compound thus obtained was converted into its hydrochloride salt as off white solid; m.p. 135-138° C.

Yield 0.75 g (68%) MS m/z 429.9 (MH⁺), IR KBr cm⁻¹ 1692.2 (C═O)., 3417 (OH)

¹H NMR (CDCl₃) δ: 1.34-1.36 (6H, d), 2.16-2.22 (4H, m), 2.57-2.62 (2H, b d), 3.15-3.21 (4H, m), 3.27-3.31 (4H, m), 3.54-3.58 (2H, m), 3.77-3.79 (2H, m), 3.98 (2H, b s), 4.51-4.59 (1H, m), 5.89 (2H, b s), 6.61-6.73 (2H, m), 6.78-6.88 (2H, m).

An illustrative list of the compounds of the invention which were synthesised by the above method is given below:
2-[N-{N′-(2-Isopropoxyphenyl)aminoethyl}acetylaminopropyl]-3a,4,7,7a-tetrahydro-1H-isoindole-1,3(2H)-dione hydrochloride; m.p. 134-137° C.,
2-[N-{N′-(2-Isopropoxyphenyl)acetylaminoethyl}aminopropyl]-3a,4,7,7a-tetrahydro-1H-isoindole-1,3(2H)-dione hydrochloride; m.p. 157-160° C.,
2-[N-{N′-(2-Isopropoxyphenyl)aminoethyl}acetaldehyde-aminopropyl]-3a,4,7,7a-tetrahydro-1H-isoindole-1,3(2H)-dione,
2-[N-{N′-(2-Isopropoxyphenyl)aminoethyl}aminopropyl-N—N′-(bishydroxyethyl]-3a,4,7,7a-tetrahydro-1H-isoindole-1,3(2H)-dione,
2-[N-{N′-(2-Isopropoxyphenyl)aminoethyl ethylacetate-aminopropyl]-3a,4,7,7a-tetrahydro-1H-isoindole-1,3(2H)-dione,
2-[N-{N′-(2-Isopropoxyphenyl)aminoethyllformylaminopropyl]-3a,4,7,7a-tetrahydro-1H-isoindole-1,3(2H)-dione,
2-[3-{4-(2-Isopropoxyphenyl)piperazin-3-oxo-1-yl{propyl]-3a,4,7,7a-tetrahydro-1H-isoindole-1,3(2H)-dione,
1-[N,N-{N′-(2-Methoxyphenyl)aminoethyl}-2-hydroxyethyl]-3-(2,6-dioxopiperidin-1-yl]aminopropane hydrochloride; m.p. 175-178° C.,
2-[3-{3-(2-Isopropoxyphenyl)imidazolidon-1-yl}propyl]-3a,4,7,7a-tetrahydro-1H-isoindole-1,3(2H)-dione,
2-[N-{N′-(2-Isopropoxyphenyl)aminoethyl}aminopropyl-N′-(β-hydroxyethyl]-3a,4,7,7a-tetrahydro-1H-isoindole-1,3(2H)-dione hydrochloride,
2-[N-{N′-(2-Isopropoxyphenyl)aminoethyl)acetylaminopropyl)-3a,4,7,7a-tetrahydro-1H-isoindole-1,3(2H)-dione.

### EXAMPLE 9

### Scheme—IX

### Preparation of 2-[3-{4-(2-Isopropoxyphenyl)-2,3-dioxopiperazin-1-yl}-propyl]-3a,4,7,7a-tetrahydro-1H-isoindole-1,3 (211)-dione;

To a solution of Compound No. 42 (Example 7) (0.5 g, 1.298 mmol) in dichloromethane was added triethylamine (0.197 g, 1.97 mmol) and the resulting reaction mixture was cooled to −10° C.; followed by dropwise addition of oxalyl chloride (0.247 g, 1.94 mmol). The reaction temperature was raised to room temperature and stirred for 1 hr. After completion of the reaction, it was quenced by adding water (10 ml) to it, and then it was extracted with ethyl acetate (2×10 ml). The combined organic layer was concentrated under reduced pressure to yield a crude oil. The crude product was purified by column chromatography on silica gel (60-120 mesh), using dichloromethane/methanol (9.8:0.2) as an eluent to afford the product as an oil.

MS m/z 440 (MH⁺), ¹H NMR (CDCl₃) δ: 1.32-1.34 (6H, d), 1.89-1.94 (2H, m), 2.17-2.25 (2H, m), 2.60-2.63 (2H, m), 3.10-3.12 (2H, m), 3.48-3.57 (4H, m), 3.64-3.67 (2H, m), 3.80-3.82 (2H, m), 4.56-4.60 (1H, m), 5.83-5.92 (2H, m), 6.87-6.98 (4H, m).

### EXAMPLE 10

### Scheme X

### Preparation of 2-[3-{4-(2-Isopropoxyphenyl)piperazin-1-yl}propyl]-4,7-diacetoxy-3a,4,7,7a-tetrahydro-1H-isoindole-1,3(2H)-dione hydrochloride;

A mixture of 1-amino-3-[4-(2-isopropoxyphenyl)piperazin-1-yl]propane (1 g, 3.6 mmol) and maleic anhydride (0.36 g, 3.6 mmol) was refluxed in toluene for 3 hrs with azeotropic removal of water. After the completion of the reaction, the solvent was removed under reduced pressure and the residue thus obtained was column chromatographed to afford an oily product (yield 0.82 g, intermediate) The mixture of this intermediate (0.8, 2.24 mmol) and 1,4-diacetoxy-1,3-butadiene (0.38 g, 2.24 mmol) were refluxed in toluene for 8 hrs. After completion of the reaction, the solvent was removed under reduced pressure. The crude product was purified by chromatography using dichloromethane methanol (9.9:0.1) as eluent. The oily product thus obtained was finally converted into its hydrochloride salt (m.p. 176-177° C.).

IR (KBr cm⁻¹): 1703.2 (C═O), 1741.3 (C═O). MS m/z: 528 (MH⁺) ¹H NMR (CDCl₃) δ: 1.35-1.37 (6H, d), 2.13 (6H, s), 2.20-2.23 (2H, m), 3.01 (4H, br s), 3.52-3.56 (6H, m), 3.61-3.63 (2H, m), 3.68-3.69 (2H, m), 4.57-4.61 (1H, m), 5.42-5.43 (2H, m), 6.16 (2H, m), 6.85-6.90 (3H, m), 6.99-7.02 (1H, m).

An illustrative list of the compounds of the invention which were synthesised by the above method is given below:
2-[3-{4-(2-Isopropoxyphenyl)piperazin-1-yl lpropyl]-5,6-dimethoxy-3a,4,7,7a-tetrahydro-1H-isoindole-1,3(2H)-dione hydrochloride; m.p. 153-155° C.,
2-[3-{4-(2-Isopropoxyphenyl)piperazin-1-yl}propyl]4,7-diphenyl-3a,4,7,7a-tetrahydro-1H-isoindole-1,3(2H)-dione hydrochloride; m.p. 193-194° C.,
2-[3-{4-(2-methoxyphenyl)piperazin-1-yl lpropyl]4,7-diphenyl-3a,4,7,7a-tetrahydro-1H-isoindole-1,3(2H)-dione hydrochloride; m.p. 224-225° C.,
2-[3-{4-(2-Isopropoxyphenyl)piperazin-1-yl}propyl]4-hydroxy-3a,4,5,6,7,7a-hexahydro-1H-isoindole-1,3(2H)-dione hydrochloride; m.p. 163-165° C.,
2-[3-{4-(2-Isopropoxyphenyl)piperazin-1-yl}propyl]-5,6-dimethoxy-3a,4,5,6,7,7a-hexahydro-1H-isoindole-1,3 (21H)-dione hydrochloride; m.p. 143-146° C.

### Pharmacological Testing Results

Receptor Binding Assays

Receptor binding assays were performed using native a-adrenoceptors. The affinity of different compounds for α_{1A} and α_{1B} adrenoceptor subtypes was evaluated by studying their ability to displace specific [³H] prazosin binding from the membranes of rat submaxillary and liver respectively (Michel et al., Br J Pharmacol, 1989: 98:883). The binding assays were performed according to U+Prichard et al. (Eur J Pharmacol, 1978; 50:87 with minor modifications.

Submaxillary glands were isolated immediately after sacrifice. The liver was perfused with buffer (Tris HCl 50 mM, NaCl 100 mM, 10 mM EDTA pH 7.4). The tissues were homogenised in 10 volumes of buffer (Tris HCl 50 mM, NaCl 100 mM, 10 mM EDTA pH 7.4). The homogenate was filtered through two layers of wet gauge and filtrate was centrifuged at 500 g for 10 min. The supernatant was subsequently centrifuged at 40,000 g for 45 min. The pellet thus obtained was resuspended in the same volume of assay buffer (Tris HCl 50 mM, 5 mM EDTA pH 7.4) and was stored at −70° C. until the time of assay.

The membrane homogenates (150-250 μg protein) were incubated in 250 μl of assay buffer (Tris HCl 50 mM, EDTA 5 mM, pH 7.4) at 24-25° C. for 1 hour. Non specific binding was determined in the presence of 300 nM prazosin. The incubation was terminated by vacuum filtration over GF/B fibre filters. The filters were then washed with ice cold 50 mM Tris HCl buffer (pH 7.4). The filtermats were dried and bound radioactivity retained on filters was counted. The IC₅₀ and Kd were estimated by using the non-linear curve-fitting program using G Pad Prism software. The value of inhibition constant Ki was calculated from competitive binding studies by using Cheng & Prusoff equation (Cheng & Prusoff, Biochem Pharmacol, 1973, 22: 3099-3108), Ki=IC₅₀/(1+L/Kd) where L is the concentration of [³H] prazosin used in the particular experiment (Table 1).

In Vitro Functional Studies

In order to study selectivity of action of these compounds towards different a-adrenoceptor subtypes, the ability of these compounds to antagonise (α_{1D}) prostate (α_{1A}) and spleen (α_{1B}) was studied. Aorta and spleen tissues were isolated from urethane anaesthetized (1.5 g/kg) male wister rats. Isolated tissues were mounted in organ bath containing Krebs Henseleit buffer of the following composition (mM): NaCl 118; KCl 4.7; CaCl₂ 2.5; MgSO₄ 7H₂O 1.2; NaHCO₃ 25; KH₂PO₄ 1.2; glucose 11.5. Buffer was maintained at 37° C. and aereated with a mixture of 95% O₂ and 5% CO₂. A resting tension of 2 g (aorta) or 1 g (spleen and prostate) was applied to tissues. Contractile response was monitored using a force displacement transducer and recorded on chart recorders. Tissues were allowed to equilibrate for 2 hours. At the end of equilibration period, concentration response curves to norepinephrine (aorta) and phenylepinephrine (spleen and prostate) were obtained in the absence and presence of tested compound (at concentration of 0.1,1 and 10 mM). Antagonist affinity was calculated and expressed as pKB values in Table II.

In Vivo Uroselectivity Study

In order to assess the uroselectivity in vivo, the effects of these compounds were studied on mean arterial pressure (MAP) and intraurethral pressure (UP) in conscious beagle dogs as per the method of Brune et al. (Pharmacol., 1996, 53:356). Briefly, male dogs were instrumented for chronic continuous measurement of arterial blood pressure by implanting a telemetry transmitter (TL11M2-D70-PCT, Data Sci. International, St. Paul, Minn. USA) into the femoral artery, two weeks prior to the study. During the recovery period, the animal was acclimatized to stay in the sling restraint. On the day of testing, overnight fasted animal was placed in the sling restraint. A Swan-Ganz. Balloon tipped catheter was introduced into the urethra at the level of prostate and the balloon was inflated (Brune. et. al. 1996). After recording the base line readings, effect of 16 μg/kg, phenylephrine (i.v.) on MAP and IUP was recorded. The response of phenylephrine to MAP and RIP were recorded at 0.5, 1, 2, 3, 4, 6, 9 and 24 hours after the oral administration of vehicle or the test drug. The changes in MAP were recorded on line using Dataquest Software (Data Sci. International, St. Paul, Minn. USA). The change in phenylephrine response on MPP administration after the test drug administration was calculated as percent change of that of control values. Area under curve was calculated, and the ratio of the values for MAP and IUP was used for calculating the uroselectivity (Table III).

TABLE I
Radioligand Binding Studies;
Affinity of compounds for Alpha-1 Adrenoceptor Subtypes

| Compound | α_{1A} | α_{1B} | |
|---|---|---|---|
| No. | (Rat Submaxillary) | (Rat Liver) | α_{1B}/α_{1A} |
| 01 | 8.55 | 80 | 9 |
| 02 | 0.17 | 27 | 159 |
| 03 | 0.26 | 47 | 181 |
| 04 | 22 | >1000 | >45 |
| 05 | 70 | 1376 | 20 |
| 06 | 38 | 263 | 7 |
| 07 | 0.56 | 106 | 189 |
| 08 | 6.6 | 4767 | 722 |
| 09 | 1068 | >1000 | |
| 10 | >1000 | >1000 | |
| 11 | 6.4 | 191 | 30 |
| 12 | 1.7 | 118 | 69 |
| 13 | 0.36 | 85 | 236 |
| 14 | 49 | 504 | 10 |
| 15 | 35 | 346 | 10 |
| 16 | 19 | 267 | 14 |
| 17 | 1.6 | 80 | 50 |
| 18 | 1.5 | 97 | 65 |
| 19 | 0.23 | 104 | 452 |
| 20 | 0.28 | 92 | 328 |
| 21 | 3.4 | 643 | 189 |
| 22 | 1587 | 1093 | 0.7 |
| 23 | 0.98 | 127 | 130 |
| 24 | 5.9 | 495 | 84 |
| 25 | 0.86 | 173 | 201 |
| 26 | 8.83 | 2090 | 237 |
| 27 | 306 | >5000 | 16 |
| 28 | 0.24 | 41 | 171 |
| 29 | 2.8 | 238 | 85 |
| 30 | 1.7 | 393 | 231 |
| 31 | 2.3 | 91 | 40 |
| 32 | 0.18 | 51 | 283 |
| 33 | 0.24 | 34 | 142 |
| 34 | 1.95 | 311 | 159 |
| 35 | 38 | 582 | 15 |
| 36 | 11 | 571 | 52 |
| 37 | 462 | >1000 | >2 |
| 38 | 141 | 760 | 5 |
| 39 | 6.9 | 1377 | 200 |
| 40 | 0.82 | 143 | 174 |
| 41 | 0.3 | 105 | 350 |
| 42 | 19 | 781 | 41 |
| 43 | 0.5 | 50 | 100 |
| 44 | 594 | 1738 | 3 |
| 45 | 8.6 | 120 | 14 |
| 46 | 379 | >1000 | >3 |
| 47 | 299 | >1000 | >3 |
| 48 | 91 | >1000 | >11 |
| 49 | >1000 | >1000 | 1 |
| 50 | 47 | >1000 | >21 |
| 51 | 662 | >15000 | >23 |
| 52 | 351 | >15000 | >43 |
| 53 | 74 | >15000 | >203 |
| 54 | 7286 | >15000 | >2 |
| 55 | 72 | 3637 | 51 |
| 56 | >100 | 992 | >10 |
| 57 | >1000 | >1000 | 1 |
| 58 | 160 | >1000 | 10 |
| 59 | 2.3 | 48 | 21 |
| 60 | 1.2 | 142 | 118 |
| 61 | 0.93 | 29 | 31 |
| 62 | >1000 | >1000 | 1 |
| 63 | >100 | >1000 | >10 |
| 64 | 28.5 | 870 | 31 |
| 65 | >1000 | >1000 | 1 |
| 66 | 5.2 | 167 | 32 |
| 67 | 189 | >10000 | >53 |
| 68 | 228.5 | >10000 | >44 |
| 69 | 7160 | >10000 | >10 |
| 70 | 6754 | 4920 | 0.7 |
| 71 | >1000 | >10000 | 1 |
| 72 | 0.54 | 142 | 263 |
| 73 | 8.45 | 192 | 23 |
| 74 | 202 | >15000 | >74 |
| 75 | 2.3 | 71 | 31 |
| 76 | 1.4 | 192 | 137 |
| 77 | 485 | 916 | 1.9 |
| 78 | 322 | 334 | 1 |

TABLE II
In Vitro Functional Assays

| Compound | α Adrenoceptor Subtype (pK_{B}) | | | Selectivity | |
|---|---|---|---|---|---|
| No. | α_{1A} | α_{1B} | α_{1D} | α_{1B}/α_{1A} | α_{1D}/α_{1A} |
| 01 | 8 | 7.42 | 7.92 | 3.8 | 1.2 |
| 02 | 9.74 | 8.89 | 10.5 | 7.07 | 0.17 |
| 03 | 9.41 | 9.56 | 9.83 | 0.7 | 0.38 |
| 04 | 8.61 | 8.15 | 7.09 | 2.9 | 33 |
| 06 | 8.18 | | 8.43 | | 0.56 |
| 07 | 8.91 | 7.8 | 8.64 | 13 | 1.9 |
| 08 | 8.38 | 8.99 | 7.66 | 0.24 | 5.24 |
| 09 | 8.15 | 7.63 | 7 | 3.3 | 14 |
| 10 | 8.83 | 7.73 | 7.23 | 13 | 40 |
| 11 | 8.14 | 9.12 | 8.43 | 0.1 | 0.5 |
| 12 | 8.78 | 7 | 8.16 | 60 | 4.2 |
| 13 | 8.49 | 7.26 | 8.64 | 17 | 0.7 |
| 17 | 9.54 | 7 | 9.07 | 347 | 3.9 |
| 18 | 9.37 | | 9.24 | | 1.3 |
| 19 | 9.1 | 7.16 | 8.57 | 87 | 3.4 |
| 20 | 9.37 | 6.99 | 8.97 | 240 | 2.5 |
| 21 | 8.33 | 7.15 | 7.61 | 15 | 5.24 |
| 23 | 8.83 | 8.13 | 8.08 | 5 | 5.6 |
| 25 | 8.34 | 7 | 8.37 | 22 | 0.93 |
| 26 | 8.8 | | 6.78 | | 105 |
| 28 | 9.01 | 7.36 | 8.85 | 45 | 1.4 |
| 29 | 9.64 | | 7.99 | | 45 |
| 30 | 8.78 | | 8.06 | | 5.2 |
| 31 | 8.84 | | 8.32 | | 3.3 |
| 32 | 9.17 | | 7.8 | | 23 |
| 33 | 9.22 | 7.96 | 8.8 | 18 | 2.6 |
| 34 | 8.9 | | 7.72 | | 15 |
| 40 | 9.47 | | 8.82 | | 4.5 |
| 41 | 9.29 | 7.17 | 8.61 | 132 | 4.8 |
| 43 | 8.77 | 7.9 | 9.13 | 7.4 | 0.43 |
| 60 | 9.44 | | 8.19 | | 18 |

TABLE III
In Vivo Uroselectivity Studies in Conscious Beagle Dogs

| | | | | | Uroselectivity |
|---|---|---|---|---|---|
| Compound | Dose | | Area Under Curve | | Ratio |
| No. | (μg/kg) | Route | MAP | IUP | IUP/MAP |
| 23 | 30 | p.o | 95 | 524 | 5.5 |
| Tamsulosin | 3 | p.o | 868 | 592 | 1.47 |
| (SR) | | | | | |

While the present invention has been described in terms of its specific embodiments, certain modifications and equivalents will be apparent to those skilled in the art and are intended to be included within the scope of the present invention.

1. A compound having the structure of Formula I: 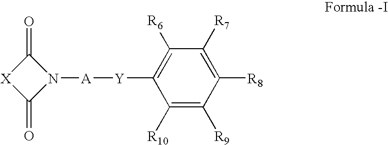
and its pharmaceutically acceptable salts, enantiomers, diastereomers, N-oxides, prodrugs, metabolities, polymorphs, or pharmaceutically acceptable solvates,
wherein X is selected from the group consisting of 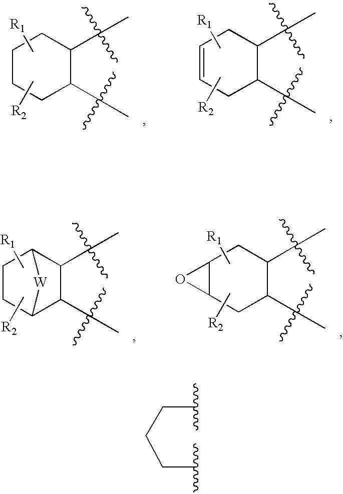
where the points of attachment are depicted by hashed bonds, and where one point of attachment is bonded to the carbonyl adjacent to the nitrogen and the second point of attachment is bonded to the other carbonyl;
W is O, S, SO or SO₂; 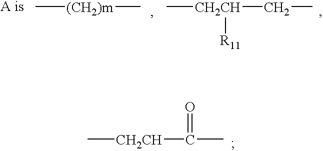
where m is one of the integers 2, 3 or 4; R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 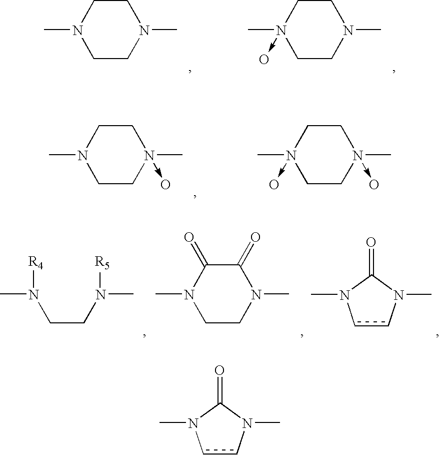
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄₎ alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₅, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 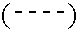
is a single bond or no bond.

2. A compound selected from the group consisting of:
1-[4-(2-Hydroxyphenyl)piperazin-1-yl]-3-(2,6-dioxopiperidin-1-yl)propane hydrochloride,
2-[3-{4-(2-(2,2,2-Trifluoroethoxy)phenyl)piperazin-1-yl}propyl]-3a,4,7,7a-tetrahydro-1H-isoindole-1,3(2H)-dione hydrochloride,
1-[4-(2-(2,2,2-Trifluoroethoxy)phenyl piperazin-1-yl]-3-(2,6-dioxopiperidin-1-yl)propanehydrochloride,
2-[3-{4-(2-Ethoxyphenyl)piperazin-1-yl, 1-N-oxide}propyl]-3a,4,7,7a-tetrahydro-1H-isoindole-1,3(2H)-dione,
1-[4-(2-Ethoxyphenyl)piperazin-1-yl]-3-(2,6-dioxopiperadin-1-yl)ethane hydrochloride,
2-[3-{4-(2-Methoxyphenyl)piperazin-1-yl}-2-hydroxypropyl]-3a,4,7,7a-tetrahydro-1H-isoindole-1,3(2H)-dione hydrochloride,
2-[3-{4-(2-Isopropoxyphenyl)piperazin-1-yl}-2-hydroxypropyl]-3a,4,7,7a-tetrahydro-1H-isoindole-1,3(2H)-dione hydrochloride,
2-[3-{4-(2-Isopropoxyphenyl)piperazin-1-yl, 1-N-oxide}propyl]-3a,4,7,7a-tetrahydro-1H-isoindole-1,3(2H)-dione,
2-[3-{4-(2-Isopropoxyphenyl)piperazin-1-yl-1,4-N,N-dioxide}propyl]-3a,4,7,7a-tetrahydro-1H-isoindole-1,3-(2H)-dione,
2-[3-{4-(2-Ethoxyphenyl)piperazin-1-yl,1,4-N,N-dioxide}propyl]-3a-4,7,7a-tetrahydro-1H-isoindole-1,3(2H)-dione,
2-[3-{4-(2-Ethoxyphenyl)piperazin-1-yl}-2-hydroxypropyl]-3a,4,7,7a-tetrahydro-1H-isoindole-1,3-(2H)-dione hydrochloride,
2-[3-{4-(2-Ethoxyphenyl)piperazin-1-yl lpropyl]-5,6-dihydroxy-3a,4,5,6,7,7a-hexahydro-1H-isoindole-1,3(2H)-dione,
2-[3-{4-(2-Isopropoxyphenyl)piperazin-1-yl}propyl]-5,6-dihydroxy-3a,4,5,6,7,7a-hexahydro-1H-isoindole-1,3(2H)-dione,
b 2-[3-{4-(2-Hydroxyphenyl)piperazin-1-yl}-2-hydroxypropyl]-3a,4,7,7a-tetrahydro-1H-isoindole-1,3(2H)-dione hydrochloride,
2-[2-{4-(2-Ethoxyphenyl)piperazin-1-yl}ethyl]-3a,4,7,7a-tetrahydro-1H-isoindole-1,3(2H)-dione hydrochloride,
2-[2-{4-(2-(2,2,2-Trifluoroethoxy)phenyl)piperazin-1-yl}ethyl]-3a,4,7,7a-tetrahydro-1H-isoindole-1,3(2H)-dione hydrochloride,
2-[3-{4-(2-Ethoxyphenyl)piperazin-1-yl lpropyl]-5-chloro-6-hydroxy-3a,4,5,6,7,7a-hexahydro-1H-isoindole-1,3(2H)-dione hydrochloride,
2-[3-{4-(2-Ethoxyphenyl)piperazin-1-yl}propyl)-5-hydroxy-3a,4,5,6,7,7a-hexahydro-1H-isoindole-1,3(2H)-dione hydrochloride,
2-[3-{4-(2-Isopropoxyphenyl)piperazin-1-yl}propyl]-5,6-epoxy-3a,4,5,6,7,7a-hexahydro-1H-isoindole-1,3(2H)-dione,
2-[3-{4-(2-Isopropoxyphenyl)piperazin-1-yl}propyl]-5-hydroxy-3a,4,5,6,7,7a-hexahydro-1H-isoindole-1,3(2H)-dione,
2-[3-{4-(2-Isopropoxy-5-hydroxyphenyl)piperazin-1-yl}propyl]-3a,4,7,7a-tetrahydro-1H-isoindole-1,3(2H)-dione hydrochloride,
2-[3-{4-(2-Hydroxyphenyl)piperazin-1-yl, 1-N-oxide}propyl]-3a,4,7,7a-tetrahydro-1H-isoindole-1,3(2H)-dione,
2-[3-{4-(2-Isopropoxyphenyl)piperazin-1-yl}propyl]-5,6-dihydroxy-3a,4,5,6,7,7a, hexahydro-1H-isoindole-1,3(2H)-dione-hydrochloride,
2-[3-{4-(2-Ethoxy-5-hydroxyphenyl)piperazin-1-yl}propyl]-3a,4,7,7a-tetrahydro-1H-isoindole-1,3-(2H)-dione hydrochloride,
2-[3-{4-(2-Isopropoxy-4-nitrophenyl)piperazin-1-yl}propyl]-3a,4,7,7a-tetrahydro-1H-isoindole-1,3(2H)-dione hydrochloride,
2-[3-{4-(2-Isopropoxy-4-aminophenyl)piperazin-1-yl}propyl]-3a,4,7,7a-tetrahydro-1H-isoindole-1,3(2H)-dione hydrochloride,
2-[3-{4-(2-isopropoxy-6-hydroxyphenyl)piperazin-1-yl}propyl]-3a,7,7a-tetrahydro-1H-isoindole-1,3(2H)-dione hydrochloride,
2-[3-{4-(2-Isopropoxyphenyl)piperazin-1-yl}propyl]-5-chloro-6-hydroxy-3a,4,5,6,7,7a-hexahydro-1H-isoindole-1,3(2H)-dione hydrochloride,
1-[4-(2-Isopropoxyphenyl)piperazin-1-yl]-2-hydroxy-3-(2,6-dioxopiperidin-1-yl)propane hydrochloride,
2-[3-{4-(2-Isopropoxyphenyl)piperazin-1-yl}-2-hydroxypropyl)-5,6-epoxy-3a,4,5,6,7,7a-hexahydro-1H-isoindole-1,3(2H)-dione,
2-[3-{4-(2-(2,2,2-Trifluoroethoxyphenyl)piperazin-1-yl}-2-hydroxypropyl]-3a,4,7,7a-tetrahydro-1H-isoindole-1,3(2H)-dione hydrochloride,
2-[3-{4-(2-(2,2,2-Trifluoroethoxy)phenyl)piperazin-1-yl lpropyl]-5,6-epoxy-3a,4,5,6,7,7a-hexahydro-1H-isoindole-1,3(2H)-dione,
2-[3-{4-(2-(2,2,2-Trifluoroethoxy)phenyl)piperazin-1-yl}propyl]-5-hydroxy-3a,4,5,6,7,7a-hexahydro-1H-isoindole-1,3(2H)-dione hydrochloride,
2-[3-{4-(2-(2,2,2-Trifluoroethoxy)phenyl)piperazin-1-yl}-2-hydroxypropyl]-5,6-epoxy-3a,4,5,7,7a-hexahydro-1H-isoindole-1,3(2H)-dione,
2-[3-{4-(2-Isopropoxy-3-hydroxyphenyl)piperazin-1-yl lpropyl]-3a,4,7,7a-tetrahydro-1H-isoindole-1,3(2H)-dione hydrochloride,
1-[4-(2-Isopropoxy-5-hydroxyphenyl)piperazin-1-yl]-3-(2,6-dioxopiperdin-1-yl)piperazin-1-yl]-3-(2,6-dioxopiperidin-1-yl)propane hydrochloride,
1-[4-(2-Isopropoxy-6-hydroxyphenyl)piperazin-1-yl]-3-(2,6-dioxopiperdin-1-yl)propane hydrochloride,
1-[4-(2-Isopropoxy-3-hydroxyphenyl)piperazin-1-yl]-3-(2,6-dioxopiperidin-1-yl)propane hydrochloride,
1-[4-{2-(2,2,2-Trifluoroethoxy)phenyl)piperazin-1-yl]-2-hydroxy-3-(2,6-dioxopiperidin-1-yl)propane hydrochloride,
2-[3-f{4-(2-Isopropoxyphenyl)piperazin-1-yl}propyl]-4-acetoxy-3a,4,7,7a-tetrahydro-1H-isoindole-1,3(2H)-dione hydrochloride,
2-[3-{4-(2-Isopropoxyphenyl)piperazin-1-yl}propyl]4-hydroxy-3a,4,7,7a-tetrahydro-1H-isoindole-1,3(2H)-dione hydrochloride,
2-[N-{N′-(2-Isopropoxyphenyl)aminoethyl)aminopropyl]-3a,4,7,7a-etrahydro-1H-isoindole-1,3(2H)-dione hydrochloride,
2-[3-{4-(2-Cyclopentyloxyphenyl)piperazin-1-yl}propyl]-3a,4,7,7a-tetrahydro-1H-isoindole-1,3(2H)-dione hydrochloride,
1-[4-(2-hydroxyphenyl)piperazin-1-yl]-2-hydroxy-3-(2,6-dioxopiperidin-1-yl]propane hydrochloride,
2-[3-{4-(2-Biphenyl)piperazin-1-yl}propyl)-3a,4,7,7a-tetrahydro-1H-isoindole-1,3(2H)-dione hydrochloride,
2-[N-{N′-(2-Isopropoxyphenyl)aminoethyl}acetylaminopropyl]-3a,4,7,7a-tetrahydro-1H-isoindole-1,3(2H)-dione hydrochloride,
2-N-{N′-(2-Isopropoxyphenyl)acetylaminoethyl}aminopropyl]-3a,4,7,7a-tetrahydro-1H-isoindole-1,3(2H)-dione hydrochloride,
2-[[N-{N′-(2-Isopropoxyphenyl)aminoethyl}hydroxyethyl]aminopropyl]-3a,4,7,7a-tetrahydro-1H-isoindole-1,3(2H)-dione hydrochloride,
1-[4-(2-Isopropoxyphenyl)piperazin-1-yl]-1-oxo-3-(2,6-dioxopiperidin-1-yl)propane hydrochloride,
2-[N-{N′-(2-Isopropoxyphenyl)aminoethyl}acetaldehyde-aminopropyl]-3a,4,7,7a-tetrahydro-1H-isoindole-1,3(2H)-dione,
2-[N-{N′-(2-Isopropoxyphenyl)aminoethyl}aminopropyl-N,N′-(bis hydroxy ethyl]-3a,4,7,7a-tetrahydro-1H-isoindole-1,3(2H)-dione,
2-[N-{N′-(2-Isopropoxyphenyl)aminoethyl}ethylacetate-aminopropyl]-3a,4,7,7a-tetrahydro-1H-isoindole-1,3(2H)-dione,
2-[N-{N′-(2-Isopropoxyphenyl) arinoethyllformylaminopropyl]-3a,4,7,7a-tetrahydro-1H-isoindole-1,3(2H)-dione,
2-[3-{4-(2-Isopropoxyphenyl)piperazin-3-oxo-1-yl}propyl]-3a,4,7,7a-tetrahydro-1H-isoindole-1,3(2H)-dione,
1-[4-(2-Methoxyphenyl)piperazin-1-yl-4-N-oxide]-3-(2,6-dioxopiperidin-1-yl]propane,
1-[N-{N′-(2-Methoxyphenyl)aminoethyl}-3-(2,6-dioxopiperidin-1-yl]aminopropane hydrochloride,
1-[N-N-{N′-(2-Methoxyphenyl)aminoethyl}]-3-(2,6-dioxopiperidin-1-yl)aminopropane hydrochloride;
2-[3-{4-(2-Isopropoxy-4-acetylaminophenyl)piperazin-1-yl}propyl]-3a,4,7,7a-tetrahydro-1H-isoindole-1,3(2H)-dione hydrochloride,
2-[3-{4-(2-Isopropoxyphenyl)piperazin-1-yl}propyl]-7,7a-dihydro-1H-isoindole-1,3(2H)-dione hydrochloride,
2-[3-{4-(2-Isopropoxyphenyl)piperazin-1-yl}-propyl]-4-hydroxy-3a,4,5,6,7,7a-hexahydro-1H-isoindole-1,3(2H)-dione hydrochloride,
2-[3-{4-(2-Isopropoxyphenyl)piperazin-1-yl}-propyl]-exo-4,7-epoxy-3a,4,7,7a-tetrahydro-1H-isoindole-1,3(2H)-dione hydrochloride,
2-[3-{4-(2-Isopropoxyphenyl)piperazin-1-yl]-1-oxo-propyl]-3a,4,7,7a-tetrahydro-1H-isoindole-1,3(2H)-dione hydrochloride,
2-[3-{4-(2-Isopropoxyphenyl)piperazin-1-yl}-1-oxo-propyl]-3a,4,5,6,7,7a-hexahydro-1H-isoindole-1,3(2H)-dione hydrochloride,
2-[3-{4-(2-Isopropoxyphenyl)piperazin-1-yl,4-N-oxide}propyl]-3a,4,7,7a-tetrahydro-1H-isoindole-1,3(2H)-dione,
2-[3-{4-(2-Isopropoxyphenyl)piperazin-1-yl,1-N-oxide}2-hydroxypropyl]-3a,4,7,7a-tetrahydro-1H-isoindole-1,3(2H)-dione,
2-[3-{4-(2-ethoxyphenyl)piperazin-1-yl}propyl)-5,6-dihydroxy-3a,4,7,7a-tetrahydro-1H-isoindole-1,3(2H)-dione hydrochloride,
2-[3-{3-(2-Isopropoxyphenyl)imidazolidon-1-yl lpropyl]-3a,4,7,7a-tetrahydro-1H-isoindole-1,3(2H)-dione,
2-[N-{N′-(2-Isopropoxyphenyl)aminoethyl}aminopropyl-N′-(β-hydroxyethyl]-3a,4,7,7a-tetrahydro-1H-isoindole-1,3(2H)-dione hydrochloride,
1-[4-(2-Methoxyphenyl)piperazin-1-yl-1-N-oxide]-3-(2,6-dioxopiperidin-1-yl]-2-hydroxypropane,
1-[4-(2-Hydroxyphenyl)piperazin-1-yl-1-N-oxide]-3-(2,6-dioxopiperidin-1-yl]propane,
2-[3-{4-(2-Isopropoxyphenyl)-2,3-dioxopiperazin-1-yl}-1-oxo-propyl]-3a,4,7,7a-tetrahydro-1H-isoindole-1,3(2H)-dione,
2-[3-{4-(2-Isopropoxyphenyl)piperazin-1-yl}propyl]4,7-dihydroxy-3a,4,7,7a-tetrahydro-1H-isoindole-1,3(2H)-dione,
2-[3-{4-(2-Isopropoxyphenyl)piperazin-1-yl}propyl]-4,7-diacetoxy-3a,4,7,7a-tetrahydro-1H-isoindole-1,3(2H)-dione hydrochloride,
2-[N-{N′-(2-Isopropoxyphenyl)aminoethyl}ethylaminopropyl]-3a,4,7,7a-tetrahydro-1H-isoindole-1,3(2H)-dione,
2-[3-{4-(2-Isopropoxyphenyl)piperazin-1-yl lpropyl]-5,6-dimethoxy-3a,4,7,7a-tetrahydro-1H-isoindole-1,3(2H)-dione hydrochloride,
2-[3-{4-(2-Isopropoxyphenyl)piperazin-1-yl}propyl]-5,6-dimethoxy-3a,4,5,6,7,7a-hexahydro-1H-isoindole-1,3(2H)-dione hydrochloride,
2-[3-{4-(2-Isopropoxyphenyl)piperazin-1-yl lpropyl]-4,7-diphenyl-3a,4,7,7a-tetrahydro-1H-isoindole-1,3(2H)-dione hydrochloride,
2-[3-{4-(2-Methoxyphenyl)piperazin-1-yl}propyl]4,7-diphenyl-3a,4,7,7a-tetrahydro-1H-isoindole-1,3(2H)-dione hydrochloride.

3. A method of selectively antagonizing α₁-adrenergic receptors in a mammal comprising administering to said mammal a therapeutically effective amount of a compound having the structure of Formula I: 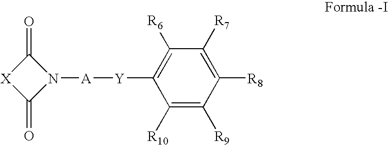
and its pharmaceutically acceptable salts, enantiomers, diastereomers, N-oxides, prodrugs, metabolities, polymorphs, or pharmaceutically acceptable solvates,
wherein X is selected from the group consisting of 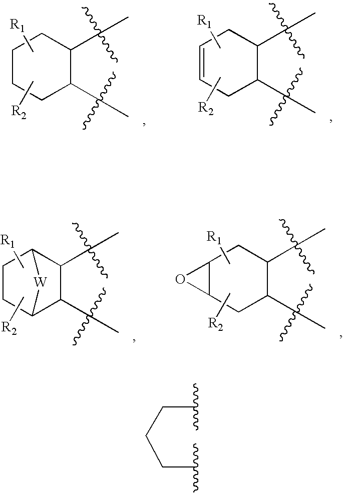
where the points of attachment are depicted by hashed bonds, and where one point of attachment is bonded to the carbonyl adjacent to the nitrogen and the second point of attachment is bonded to the other carbonyl;
W is O, S, SO or SO₂; 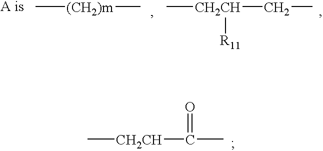
where m is one of the integers 2, 3 or 4; R₁₁, is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 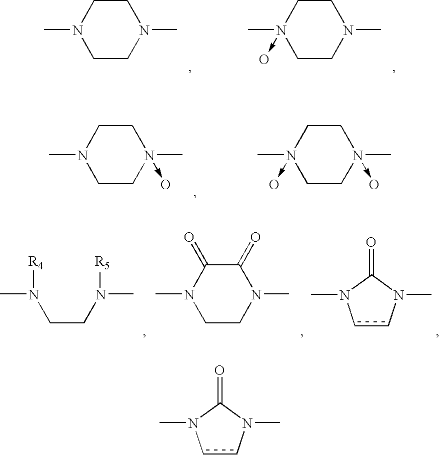
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alky, lower (C₁₋₄) alkoxy, lower (C₁₋₄) alkylthio, lower (C₁₋₄) perhaloalky, lower (C₁₋₄) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, P, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 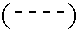
is a single bond or no bond.

4. A method of treating benign prostatic hyperplasia in a mammal comprising administering to said mammal a therapeutically effective amount of a compound having the structure of Formula I: 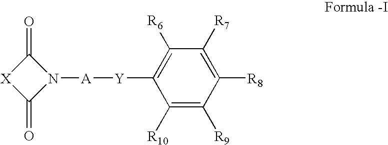
and its pharmaceutically acceptable salts, enantiomers, diastereomers, N-oxides, prodrugs, metabolities, polymorphs, or pharmaceutically acceptable solvates,
wherein X is selected from the group consisting of 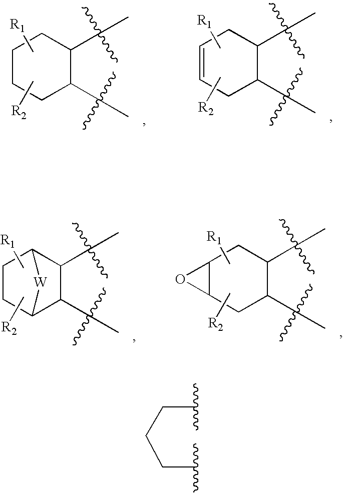
where the points of attachment are depicted by hashed bonds, and where one point of attachment is bonded to the carbonyl adjacent to the nitrogen and the second point of attachment is bonded to the other carbonyl;
W is O, S, SO or SO₂; 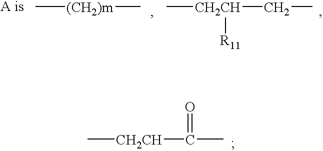
where m is one of the integers 2, 3 or 4; R₁₁, is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 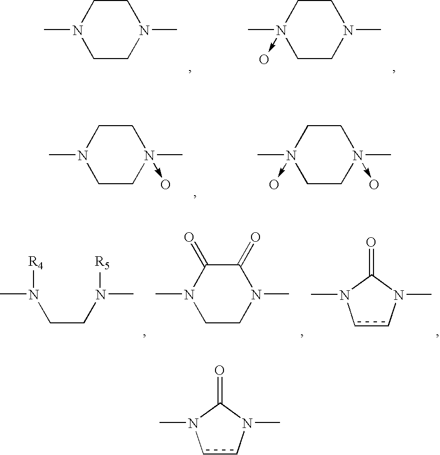
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy;
lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 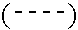
is a single bond or no bond.

5. A pharmaceutical composition comprising a therapeutically effective amount of a compound as defined in claim 1 or 2 and a pharmaceutical acceptable carrier.

6. A method of selectively antagonizing α₁-adrenergic receptors in a mammal comprising the step of administering to said mammal a therapeutically effective amount of the pharmaceutical composition according to claim 5.

7. A method for treating benign prostatic hyperplasia in a mammal comprising the step of administering to said mammal a therapeutically effective amount of the pharmaceutical composition according to claim 5.

8. A process for preparing a compound of Formula I, and its pharmaceutically acceptable salts, enantiomers, diastereomers, N-oxides, prodrugs, metabolities, polymorphs, or pharmaceutically acceptable solvates, which comprises reacting of compound of Formula II with a compound of Formula III as shown below: 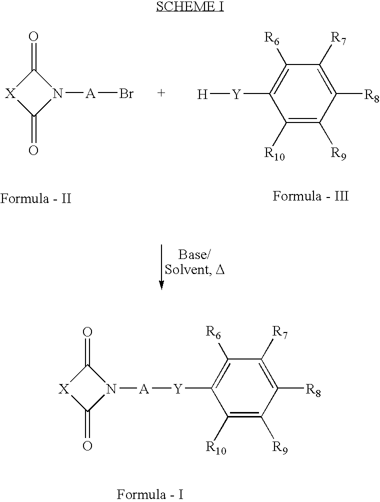
wherein X is selected from the group consisting of 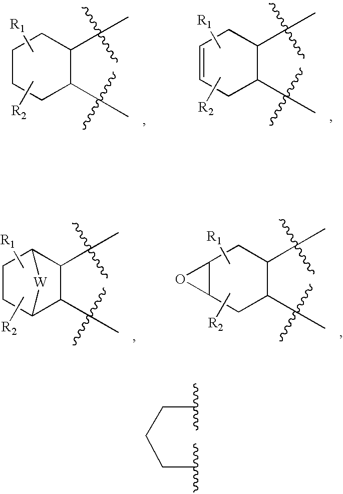
where the points of attachment are depicted by hashed bonds, and where one point of attachment is bonded to the carbonyl adjacent to the nitrogen and the second point of attachment is bonded to the other carbonyl;
W is O, S, SO or SO₂; 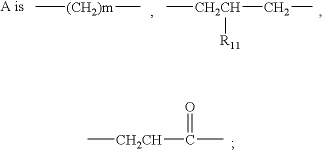
where m is one of the integers 2, 3 or 4; R₁₁, is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 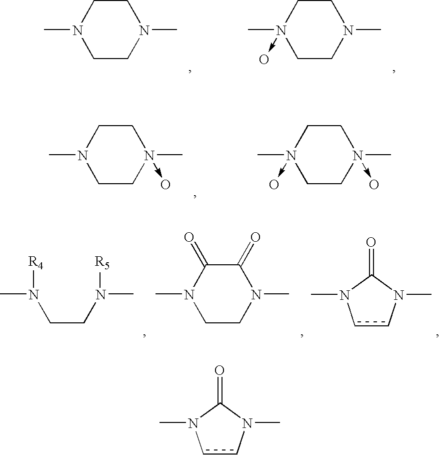
R₁ and R₂ are independently selected from H, OH, CN, NO₂, Cl, F, Br, I, OR₃, COR₃, OCOR₃, COOR₃, NH₂, N (R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 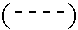
is a single bond or no bond.

9. The process of claim 8 wherein the reaction of compound of Formula II and Formula III is carried out in a suitable dipolar aprotic solvent, wherein the solvent is selected from the group consisting of dimethylsulfoxide, N N-dimethyl formamide, sulfolane, dimethylacetamide, hexamethyl phosphoramide and N-methyl-2-pyrrolidone.

10. The process of claim 8 wherein the reaction of compound of Formula II and m is carried out in the presence of a suitable inorganic base wherein the base is selected from the group consisting of sodium hydride, cesium carbonate, potassium carbonate and sodium carbonate.

11. A process for preparing a compound of Formula I, and its pharmaceutically acceptable salts, enantiomers, diastereomers, N-oxides, prodrugs, metabolites, polymorphs, or pharmaceutically acceptable solvates thereof, which comprises reacting a compound of Formula IV with a compound of Formula V as shown below: 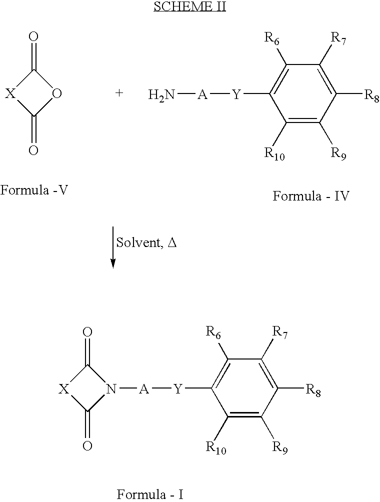
wherein X is selected from the group consisting of 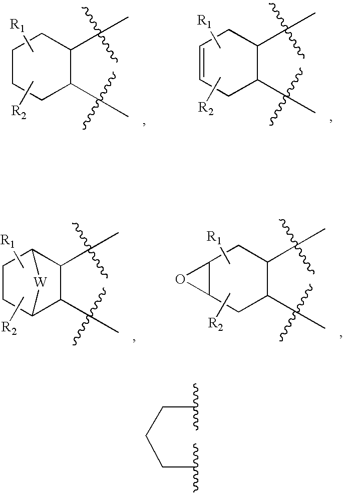
where the points of attachment are depicted by hashed bonds, and where one point of attachment is bonded to the carbonyl adjacent to the nitrogen and the second point of attachment is bonded to the other carbonyl;
W is O, S, SO or SO₂; 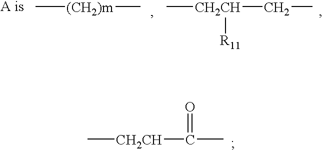
where m is one of the integers 2, 3 or 4; R₁₁, is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 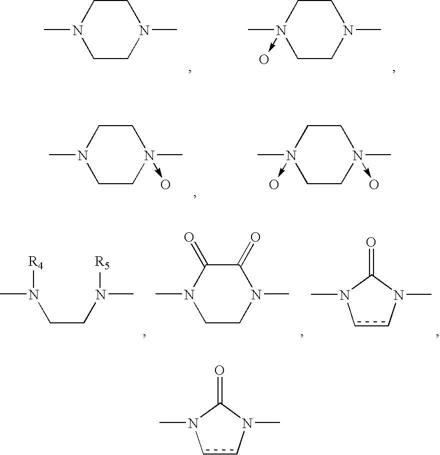
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N, N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂ (C₁₋₄) alkyl or (C₁₋₄) alkoxy (C₁₋₄) perhaloalkyl, (C₁₋₄) perhaloalkoxy wherein the broken line 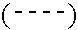
is a single bond or no bond.

12. The process of claim 11 wherein the reaction of Formula IV and Formula V is carried out in an organic solvent selected from the group consisting of benzene, toluene, xylene, pyridine, and mixture(s) thereof.

13. A process for preparing a compound of Formula I, and its pharmaceutically acceptable salts, enantiomers, diastereomers, N-oxides, prodrugs, metabolites, polymorphs, or pharmaceutically acceptable solvates thereof, which comprises reacting a compound of Formula III with a compound of Formula VI, as below: 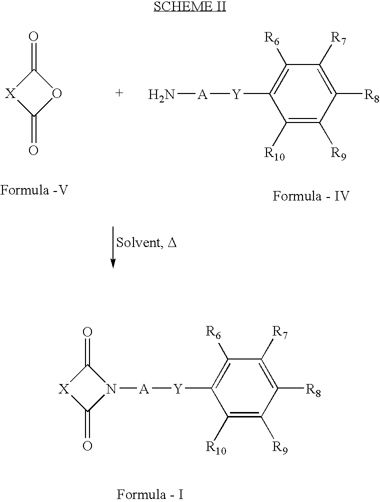
wherein X is selected from the group consisting of 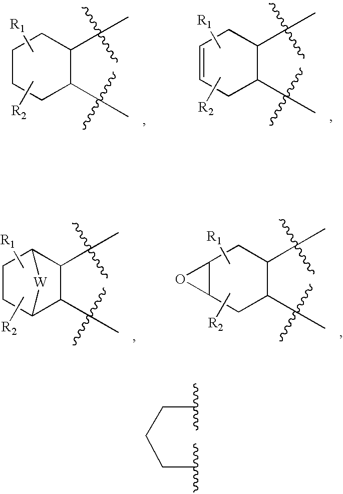
where the points of attachment are depicted by hashed bonds, and where one point of attachment is bonded to the carbonyl adjacent to the nitrogen and the second point of attachment is bonded to the other carbonyl;
W is O, S, SO or SO₂; 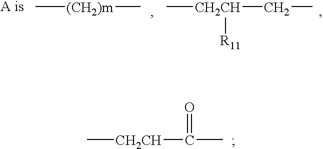
where m is one of the integers 2, 3 or 4; R₁, is independently selected from H, F, Cl, Br, I, OH, straight or branched lower (C₁₋₆) alkyl, lower (C₁₋₆)alkoxy, lower (C₁₋₆) perhaloalkyl, lower (C₁₋₆) perhaloalkoxy;
Y is selected from the group consisting of 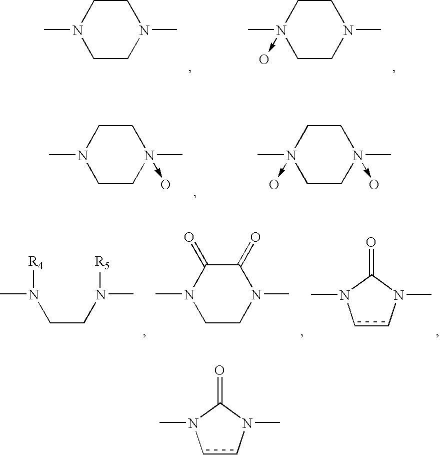
R₁ and R₂ are independently selected from H, OH, CN, NO₂, Cl, F, Br, I, OR₃, COR₃, OCOR₃, COOR₃, NH₂, N (R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 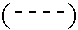
is a single bond or no bond.

14. The process of claim 13 wherein the reaction of the compound of Formula VI and Formula III is carried out in a suitable solvent to give compounds of Formula I, wherein the solvent is selected from the group consisting of dimethylsulfoxide, N,N-dimethyl formamide, sulfolane, dimethylacetamide, hexamethyl phosphoramide, N-methyl-2-pyrrolidone, and ethanol.

15. The process of claim 13 wherein the reaction of compound of Formula III and Formula VI is carried out in the presence of a base, wherein the base is selected from the group consisting of potassium carbonate, cesium carbonate, sodium carbonate, triethylamine, and diisopropylamine.

16. A process for preparing a compound of Formula IX (Formula I, when 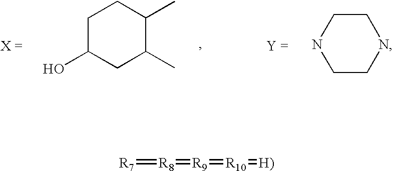
and its pharmaceutically acceptable salts, enantiomers, diastereomers, N-oxides, prodrugs, metabolites, polymorphs, or pharmaceutically acceptable solvates thereof which comprises epoxidizing the compound of Formula II to give a compound of Formula VII, which is further reacted with a compound of Formula III 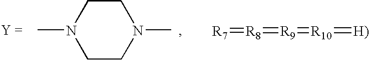
to yield a compound of Formula VII which on catalytic hydrogenation gives a compound of Formula IX as shown below:

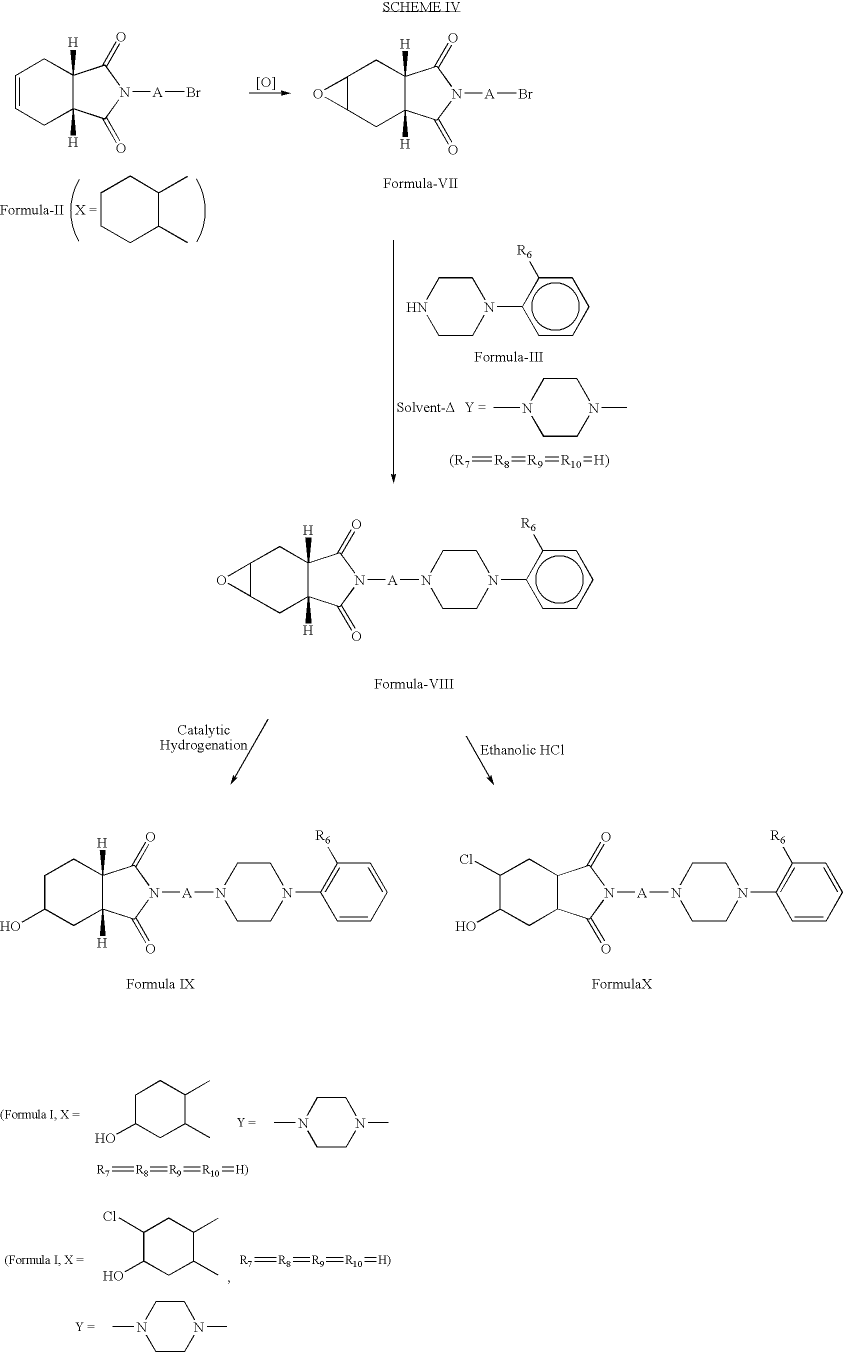

17. The process of claim 16 wherein the epoxidation of compound of Formula II is carried out with a suitable peracid, wherein the peracid is selected from the group consisting of metachloroperbenzoic acid, peracetic acid, and trifluoroperacetic acid.

18. The process of claim 16 wherein the epoxidation of compound of Formula II is carried out in a suitable solvent wherein the solvent is selected from the group consisting of dichloromethane, dichloroethane, chloroform, tetrahydrofuran, acetone, and acetonitrile.

19. The process of claim 16 wherein the reaction of epoxide intermediate of Formula VII and compound of Formula III to give compound of Formula VIII is carried out in a suitable solvent wherein the solvent is selected from the group consisting of dimethylsulfoxide, N,N-dimethylformamide, sulfolane, dimethylacetamide, hexamethyl phosphoramide, and N-methyl-2-pyrrolidone.

20. The process of claim 16 wherein the reaction of the epoxide intermediate of Formula VII and a compound of Formula III is carried out in the presence of a suitable base wherein the base is selected from the group consisting of sodium hydride, cesium carbonate, potassium carbonate, and sodium carbonate.

21. The process of claim 16 wherein catalytic hydrogenation of compound of Formula VIII to give compound of Formula IX is carried out in a suitable solvent, wherein the solvent is selected from the group consisting of methanol and ethanol.

22. The process of claim 16 wherein the compound of Formula VIII on nucleophilic epoxide ring opening with alcoholic hydrochloric acid gives a compound of Formula X (Formula I, when

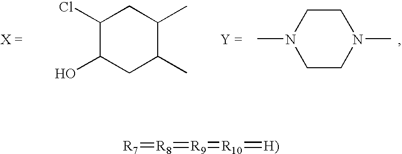

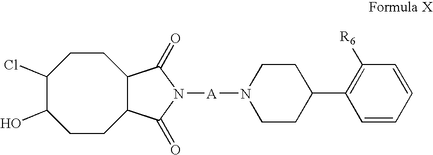

23. A process for preparing a compound of Formula XII (Formula I, 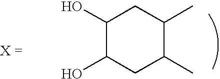
and its pharmaceutically acceptable salts, enantiomers, diastereomers, N-oxides, prodrugs, metabolites, polymorphs, or pharmaceutically acceptable solvates thereof, which comprises reacting a compound of Formula XI (Formula I, 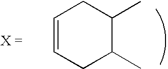
with an oxidising agent to give a compound of Formula XII as shown below:

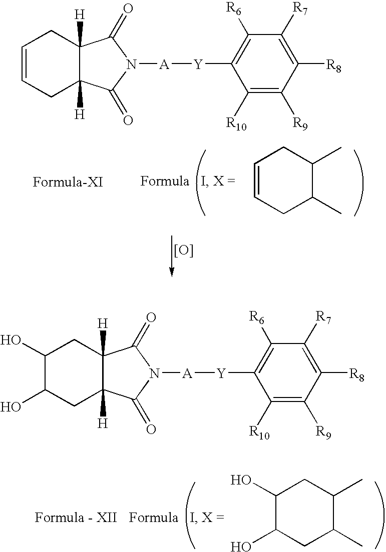

24. The process of claim 23 wherein the reaction of compound of Formula XI with an oxidising agent is carried out in a solvent selected from the group consisting of methanol, ethanol, acetone, and acetonitrile.

25. The process of claim 23 wherein a compound of Formula XI is oxidised to a compound of Formula XII with an oxidising agent selected from the group consisting of osmium tetraoxide and potassium permanganate.

26. A process for preparing a compound of Formula XV (Formula I, 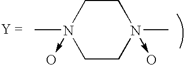
and its pharmaceutically acceptable salts, enantiomers, diastereomers, N-oxides, prodrugs, metabolites, polymorphs, or pharmaceutically acceptable solvates thereof, comprising oxidising a compound of Formula XV (Formula I, 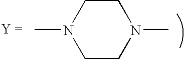
with a peracid as shown below: 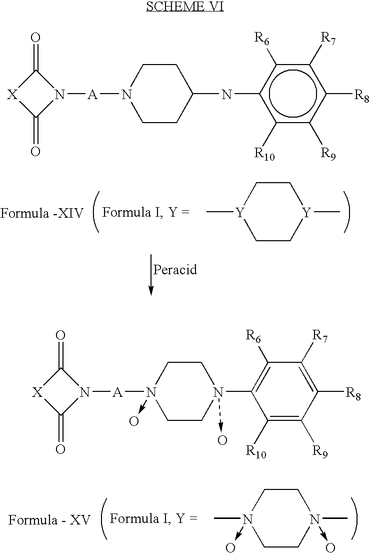
wherein X is selected from the group consisting of 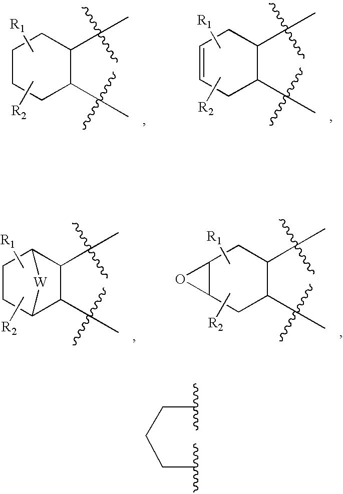
where the points of attachment are depicted by hashed bonds, and where one point of attachment is bonded to the carbonyl adjacent to the nitrogen and the second point of attachment is bonded to the other carbonyl;
W is O, S, SO or SO₂; 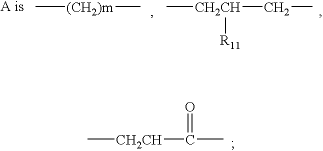
where m is one of the integers 2, 3 or 4; R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 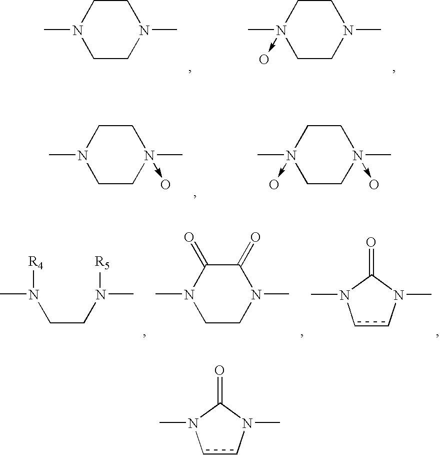
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of P, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 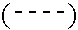
is a single bond or no bond.

27. A process for preparing a compound of Formula XVII (Formula I, wherein 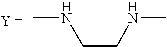
) and its pharmaceutically acceptable salts, enantiomers, diastereomers, N-oxides, prodrugs, metabolites, polymorphs, or pharmaceutically acceptable solvates thereof, comprising condensing α,ω-dicarboximides of Formula II with ethylene diamines of formula XVI as shown below: 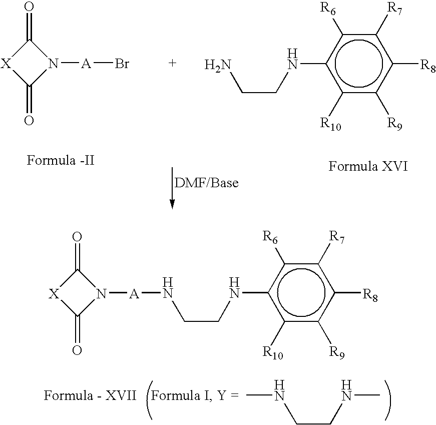
wherein X is selected from the group consisting of 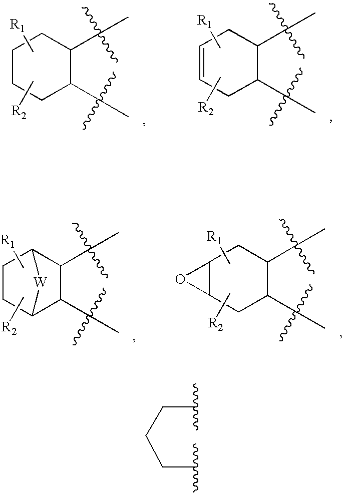
where the points of attachment are depicted by hashed bonds, and where one point of attachment is bonded to the carbonyl adjacent to the nitrogen and the second point of attachment is bonded to the other carbonyl;
W is O, S, SO or SO₂; 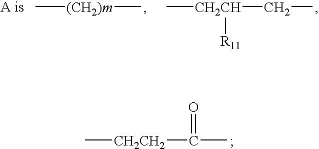
where m is one of the integers 2, 3 or 4; R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 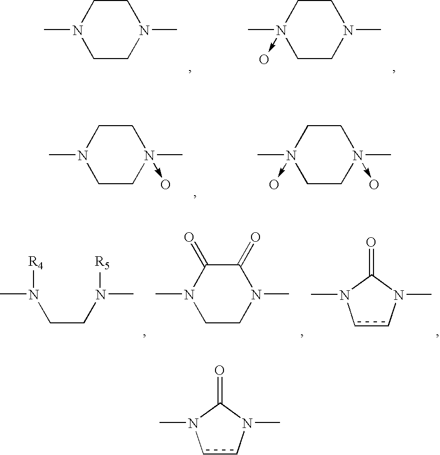
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₄) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 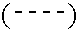
is a single bond or no bond.

28. The process of claim 27 wherein the reaction of compound of Formula II and Formula XVI is carried out in the presence of a suitable base, wherein the base is selected from the group consisting of sodium carbonate and potassium carbonate.

29. The process of claim 27 wherein the reaction of compound of Formulae II and XVI is carried out in the presence of a solvent selected from the group consisting of dimethylsulfoxide, N,N-dimethylformamide, sulfolane, dimethylacetamide, hexamethyl phosphoramide, and N-methyl-2-pyrrolidone.

30. A process of preparing of Formula XIX (Formula I, when 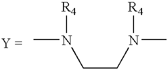
) and its pharmaceutically acceptable salts, enantiomers, diastereomers, N-oxides, prodrugs, metabolites, polymorphs, or pharmaceutically acceptable solvates thereof, comprising alkylating a compound of Formula XVIII as shown below: 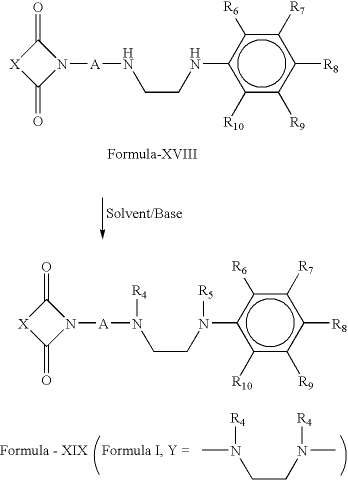
wherein X is selected from the group consisting of 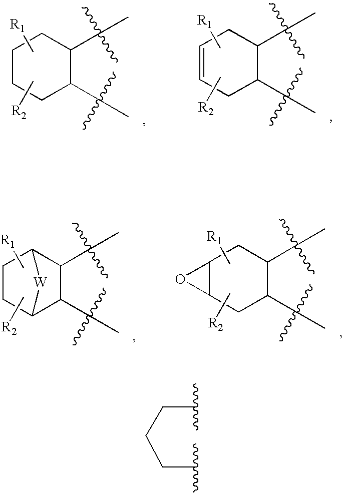
where the points of attachment are depicted by hashed bonds, and where one point of attachment is bonded to the carbonyl adjacent to the nitrogen and the second point of attachment is bonded to the other carbonyl;
W is O, S, SO or SO₂; 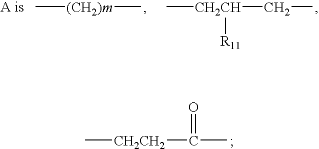
where m is one of the integers 2, 3 or 4; R₁₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 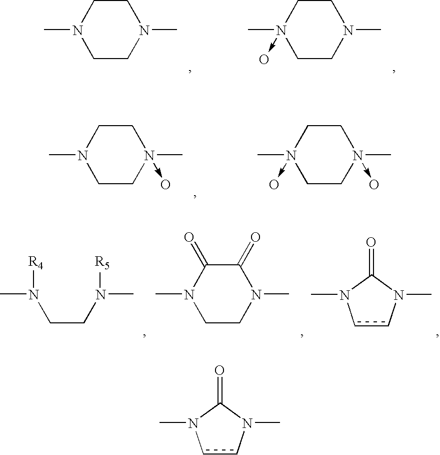
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of P, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 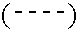
is a single bond or no bond.

31. The process of claim 30 wherein a compound of Formula XVIII is alkylated in a suitable organic solvent wherein the solvent is selected from the group consisting of dimethylsulfoxide, N,N-dimethylformamide, sulfolane, dimethylacetamide, hexamethyl phosphoramide, and N-methyl-2-pyrrolidone.

32. The process of claim 30 wherein the alkylation is carried out in the presence of an inorganic base selected from the group consisting of potassium carbonate, sodium carbonate, and sodium hydride.

33. A process for preparing a compound of Formula XX (Formula I, when 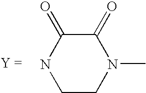
) and its pharmaceutically acceptable salts, enantiomers, diastereomers, N-oxides, prodrugs, metabolites, polymorphs, or pharmaceutically acceptable solvates thereof, comprising reacting a compound of Formula XVIII with oxalyl chloride as shown below: 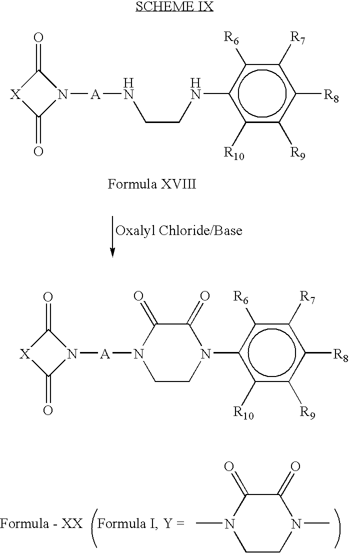
wherein X is selected from the group consisting of 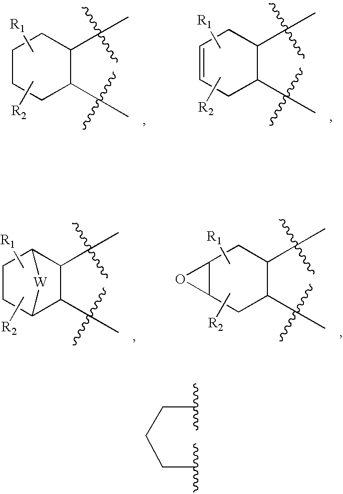
where the points of attachment are depicted by hashed bonds, and where one point of attachment is bonded to the carbonyl adjacent to the nitrogen and the second point of attachment is bonded to the other carbonyl;
W is O, S, SO or SO₂; 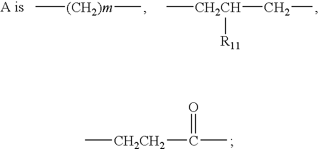
where m is one of the integers 2, 3 or 4; R₁₁, is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 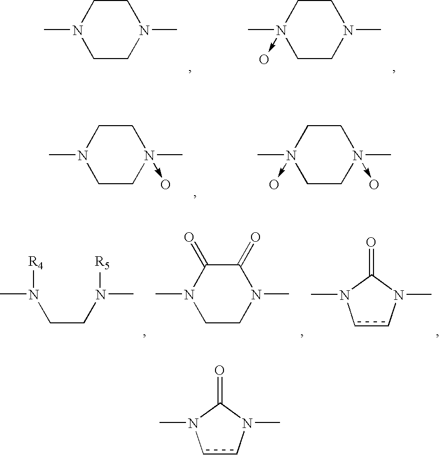
R₁ and R₂ are independently selected from H, OH, CN, NO₂, Cl, F, Br, I, OR₃, COR₃, OCOR₃, COOR₃, NH₂, N(R₄, R₅), lower (C-4) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, N(C₁₋₄) alkyl or (C₁₋₄) alkoxy (C₁₋₄) perhaloalkyl, (C₁₋₄) perhaloalkoxy wherein the broken line 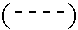
is a single bond or no bond.

34. The process of claim 33 wherein Formula XVIII is converted to its dioxo analog of Formula XX upon treatment with oxalyl chloride in the presence of a suitable organic base wherein the base is selected from the group consisting of triethylamine and diisopropyl ethylamine.

35. The process of claim 33 wherein the reaction of compound of Formula XVIII is carried out to a compound of Formula XX with oxalyl chloride in a suitable organic solvent wherein the solvent is selected from the group consisting of dichloromethane, dichloroethane, chloroform, and tetrahydrofuran.

36. A process for preparing a compound Formula XXII (Formula I, when 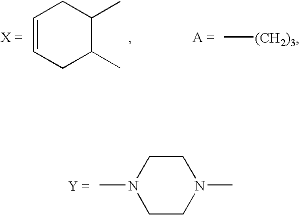
) and its pharmaceutically acceptable salts, enantiomers, diastereomers, N-oxides, prodrugs, metabolites, polymorphs, or pharmaceutically acceptable solvates thereof, comprising condensing maleic anhydride with substituted phenylpiperazine of Formula IV 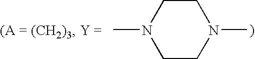
as shown below: 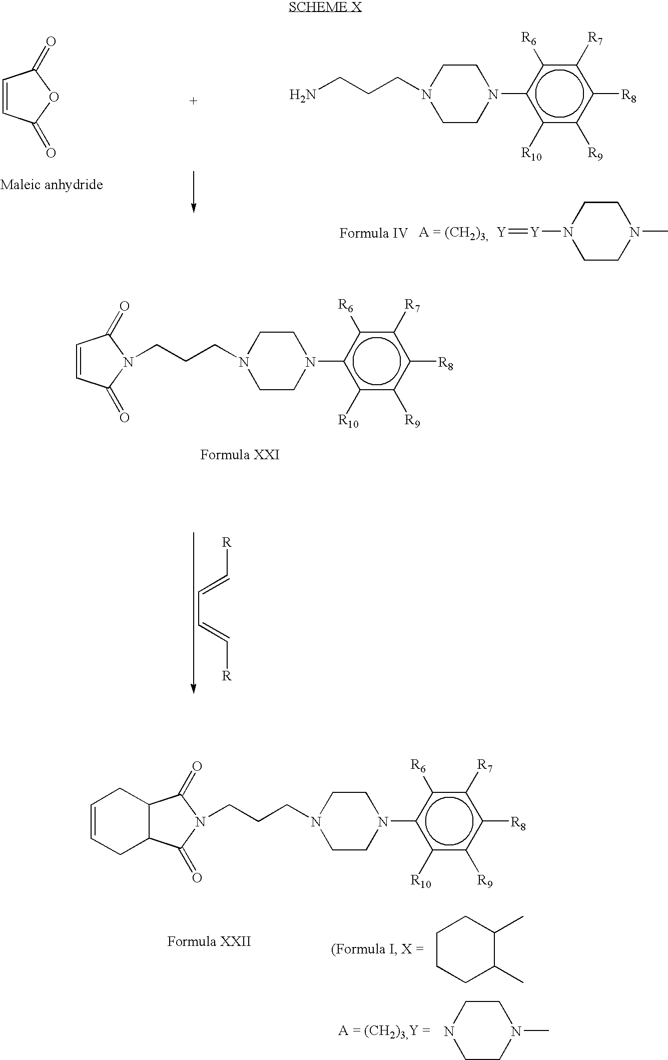
wherein X is selected from the group consisting of 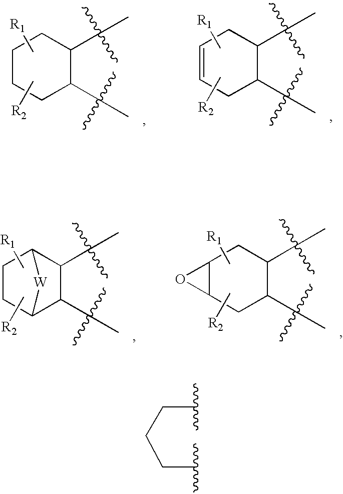
where the points of attachment are depicted by hashed bonds, and where one point of attachment is bonded to the carbonyl adjacent to the nitrogen and the second point of attachment is bonded to the other carbonyl;
W is O, S, SO or SO₂; 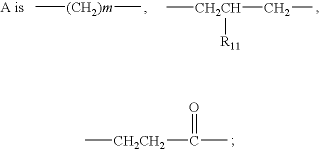
where in is one of the integers 2, 3 or 4; R₁ is independently selected from H, F, Cl, Br, I, OH, straight or branched lower (C₁₋₆) alkyl, lower (C₁₋₆) alkoxy, lower (C₁₋₆) perhaloalkyl, lower (C₁₋₆) perhaloalkoxy;
Y is selected from the group consisting of 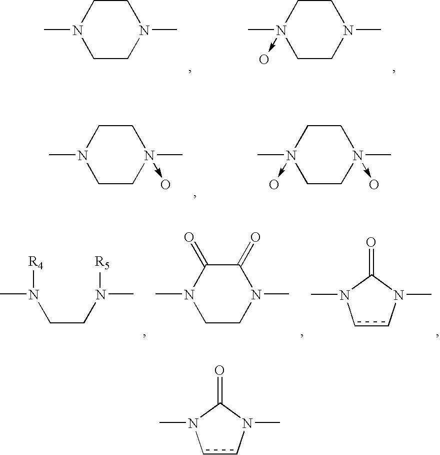
R₁ and R₂ are independently selected from H, OH, CN, NO₂, Cl, F, Br, I, OR₃, COR₃, OCOR₃, COOR₃, NH₂, N(R₄, R₅), lower (C₁₋₄) alkyl, lower (C₁₋₄) alkoxy, lower (C₁₋₄) alkylthio, lower (C₁₋₄) perhaloalkyl, lower (C₁₋₆) perhaloalkoxy; lower (C₁₋₄) alkoxy substituted with one or more of F, Cl, Br, I, OH, OR₃ or optionally substituted groups selected from aryl, aryloxy, aralalkyl, heterocyclyl or heteroaryl the said substituents being H, F, Cl, Br, I, OH, OR₃, lower (C₁₋₄) alkyl, lower (C₁₋₄) alkyl substitued with one or more of F, Cl, Br, I, OH or OR₃, wherein R₃, is selected from the group consisting of H, straight or branched C₁-C₆ alkyl or perhaloalkyl; R₄ and R₅ are independently selected from the group consisting of H, CHO, substituted or unsubstituted lower (C₁₋₄) alkyl, lower (C₁₋₄) alkoxy, COR₃, COOR₃, CH₂CH(OR₃)₂, CH₂COOR₃, CH₂CHO or (CH₂)₂OR₃ where R₃ is the same as defined above; R₆, R₇, R₈, R₉ and R₁₀ are independently selected from H, OH, CN, NO₂, Cl, F, Br, I, straight or branched lower (C₁₋₄) alkyl optionally substituted with one or more halogens, lower (C₁₋₄) alkoxy optionally substituted with one or more halogens, (C₃₋₆) cycloalkoxy, NH₂, N-lower (C₁₋₄) alkylamino, N,N-di-lower (C₁-C₄) alkylamino, N-lower alkyl(C₁-C₄)amino carbonyl, hydroxy substituted with aromatic or non-aromatic five or six membered ring, phenyl, phenyl substituted by Cl, F, Br, I, NO₂, NH₂, (C₁₋₄) alkyl or (C₁₋₄) alkoxy (C₁₋₄) perhaloalkyl, (C₁₋₄) perhaloalkoxy wherein the broken line 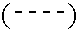
is a single bond or no bond.
